(19) United States
(12) Patent Application Publication
(10) Pub. No.: US 2021/0353687 A1
(43) Pub. Date: Nov. 18, 2021

(54) METHODS AND COMPOSITIONS FOR TREATING SUBJECTS EXPOSED TO VESICANTS AND OTHER CHEMICAL AGENTS

(71) Applicant: PLURISTEM LTD., Haifa (IL)

(72) Inventors: Arie EISENKRAFT, Tel Mond (IL); Yaacob YANAY, Shimshit (IL); Rachel OFIR, Adi (IL)

(73) Assignee: PLURISTEM LTD., Haifa (IL)

(21) Appl. No.: 17/268,795

(22) PCT Filed: Jun. 18, 2019
(86) PCT No.: PCT/IB2019/055074

§ 371 Date: Feb. 16, 2021

Related U.S. Application Data
(60) Provisional application No. 62/723,026, filed on Aug. 27, 2018.

(51) Int. Cl. A61K 35/50; C12N 5/073; A61P 39/00

(52) U.S. Cl. CPC A61K 35/50; A61P 39/00; C12N 5/0605

(57) ABSTRACT

Disclosed herein are methods and compositions comprising adherent stromal cells.

### FIELD

Disclosed herein are methods and compositions comprising placental-derived adherent stromal cells.

### BACKGROUND

Vesicants (blister agents) are chemical compounds that may cause skin, eye and/or mucosal pain and irritation. They may cause severe chemical burns, resulting in painful blisters on the bodies of those affected. Although the term is often used in connection with chemical spills and chemical warfare agents, some naturally occurring substances are also vesicants. Vesicants include mustard compounds and Lewisite. These relatively short-term effects of vesicant exposure are usually manifest within 24-48 hours of exposure. Exposure to vesicants also causes long-term effects, including pulmonary, gastrointestinal, hematological manifestations. Even if the short-term effects are alleviated with standard medical treatment or are otherwise not lethal, the long-term effects may result in significant morbidity and mortality, and require countermeasures.

Cholinesterase inhibitors, including acetylcholinesterase inhibitors (e.g. nerve agents) and butyrylcholinesterase inhibitors, are compounds that cause cholinergic syndrome, including miosis with darkness and narrowing of the visual field, ocular pain, lacrimation, nausea, vomiting, headache, rhinorrhea, dyspnea, excessive sweating, muscle fasciculation and twitching, loss of consciousness, seizures, convulsions, and respiratory failure. In certain cases, subjects exhibit cardiac arrhythmias, atrioventricular block and cardiac arrest (with high-dose exposure) and hypotension. Long-term symptoms include mainly CNS damage, which may manifest as ataxia, epilepsy, learning difficulties, memory and sleep disturbances. Even if the short-term effects are not lethal, the long-term effects may result in significant morbidity and mortality, and require countermeasures.

### SUMMARY

In one embodiment, there is provided a method of mitigating damage or morbidity following exposure to a vesicant, comprising the step of administering to the subject a pharmaceutical composition comprising adherent stromal cells (ASC), thereby mitigating damage or morbidity. In certain embodiments, the ASC are derived from a placenta, from adipose tissue, or from bone marrow (BM). Alternatively or in addition, the damage or morbidity is long-term damage or morbidity.

In another embodiment, there is provided a method of mitigating damage or morbidity following exposure to a cholinesterase inhibitor, comprising the step of administering to the subject a pharmaceutical composition comprising ASC, thereby mitigating damage or morbidity. In certain embodiments, the ASC are derived from a placenta, adipose tissue, or BM. Alternatively or in addition, the damage or morbidity is long-term damage or morbidity. Non-limiting embodiments of cholinesterase inhibitors include butyrylcholinesterase inhibitors and acetylcholinesterase inhibitors, more specifically organophosphorus agents and carbamates.

In another embodiment, there is provided a method of mitigating damage or morbidity following exposure to a chemical agent, comprising the step of administering to the subject a pharmaceutical composition comprising ASC, thereby mitigating damage or morbidity. In certain embodiments, the ASC are derived from placenta, adipose tissue, or BM. Alternatively or in addition, the damage or morbidity is long-term damage or morbidity.

In certain embodiments, the ASC described herein have been cultured on a 2-dimensional (2D) substrate, a 3-dimensional (3D) substrate, or a combination thereof. Non-limiting examples of 2D and 3D culture conditions are provided in the Detailed Description and in the Examples.

Reference herein to “growth” of a population of cells is intended to be synonymous with expansion of a cell population. In certain embodiments, ASC (which may be, in certain embodiments, placental ASC), are expanded without substantial differentiation. In various embodiments, the described expansion is on a 2D substrate, on a 3D substrate, or a 2D substrate, followed by a 3D substrate.

Except where otherwise indicated, all ranges mentioned herein are inclusive.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram of a bioreactor that can be used to prepare the cells.

FIG. 2 contains pictures of BM-derived MSC (top row) or placental ASC after adipogenesis assays. Cells were incubated with (left column) or without (right column) differentiation medium. Placental ASC were expanded in SRM (middle 3 rows depict 3 different batches) or in full DMEM (bottom row).

FIG. 3 contains pictures of BM-derived MSC (top row) or placental ASC after osteogenesis assays. Cells were incubated with (left column) or without (right column) differentiation medium. Placental ASC were expanded in SRM (middle 3 rows depict 3 different batches) or in full DMEM (bottom row).

FIG. 4 is a graph showing the survival (vertical axis) of placental ASC exposed to various concentrations (horizontal axis) of sulfur mustard.

FIG. 5 is a graph showing the survival (vertical axis) of mice exposed to no treatment (dots); or exposed to sulfur mustard, followed by no treatment (alternating 2 dashes+dot), or treatment with placebo (short dashes) or placental ASC 4 and 72 hrs. (alternating 1 dash+dot) or 24 and 72 hrs. (long dashes) afterwards.

FIG. 6A is a graph showing levels (vertical axis) of selected human proteins (horizontal axis) secreted by fetal and placental ASC populations (n=4). Units are thousands of picograms per milliliter (μg/ml) and μg/ml in the left and right panels, respectively. Left panel shows G-CSF, GRO, IL-6, IL-8, MCP-1, and ENA-78; right panel shows GM-CSF, fractalkine, MCP-3, and LIF. B is a graph showing induction of BM cell migration by maternal and fetal CM (middle and right bars, respectively), relative to SDF-1 (positive control; left bar). Units are number of colonies, after subtracting negative control (vertical axis).

FIG. 7A is a perspective view of a carrier (or “3D body”), according to an exemplary embodiment. B is a perspective view of a carrier, according to another exemplary embodiment. C is a cross-sectional view of a carrier, according to an exemplary embodiment.

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aspects of the invention relate to methods and compositions that comprise adherent stromal cells (ASC). In some embodiments, the ASC are derived from placenta, while in other embodiments, the ASC are derived from adipose tissue. Alternatively or in addition, the ASC may be human ASC, or in other embodiments animal ASC.

In some embodiments, there is provided a method of reducing a morbidity in a subject exposed to a vesicant, comprising the step of administering to the subject a pharmaceutical composition comprising ASC, thereby reducing a morbidity in a subject exposed to a vesicant.

The vesicant is, in some embodiments, a mustard compound, while in other embodiments, it is another vesicant; e.g. another vesicant known in the art. As provided herein, ASC are capable of reducing the incidence of morbidity following exposure to vesicants (as exemplified with fetal/placental cells and sulfur mustard), as well as prophylactically ameliorating long-term effects of exposure to vesicants. In more specific embodiments, the aforementioned morbidity may include chronic inflammation, pulmonary fibrosis, pulmonary hypertension, pancytopenia, chronic ocular injuries (e.g. neovascularization on the cornea, which in some embodiments leads to blindness), pathological wound healing (e.g. pathological scar tissue formation), or any combination thereof, each of which represents a separate embodiment.

In other embodiments, there is provided a method of reducing mortality in a subject exposed to a vesicant, comprising the step of administering to the subject a pharmaceutical composition comprising ASC, thereby reducing mortality in a subject exposed to a vesicant. As provided herein, ASC are capable of reducing the incidence of mortality following exposure to vesicants. In more specific embodiments, the mortality may result from chronic inflammation, pulmonary fibrosis, pulmonary hypertension, pancytopenia, pathological wound healing (e.g. pathological scar tissue formation), or complications thereof, each of which represents a separate embodiment.

In some embodiments, there is provided a method of reducing pulmonary symptoms of exposure to a vesicant, such as chronic inflammation, pulmonary fibrosis, pulmonary hypertension, and bronchiolitis obliterans syndrome (BOS) (Gronningszter I S et at), comprising the step of administering to the subject a pharmaceutical composition comprising ASC, thereby reducing pulmonary symptoms in a subject exposed to a vesicant. The vesicant is, in some embodiments, a mustard compound, while in other embodiments, it is another vesicant; e.g. another vesicant known in the art. As provided herein, ASC are capable of reducing the incidence of pulmonary symptoms following exposure to vesicants, as well as prophylactically ameliorating long-term effects of exposure to vesicants.

In some embodiments, there is provided a method of reducing hematological symptoms of exposure to a vesicant, such as anemia, bleeding/hemorrhage, bone marrow suppression, increased susceptibility to infection, leukocytopenia, and thrombocytopenia, comprising the step of administering to the subject a pharmaceutical composition comprising ASC, thereby reducing hematological symptoms in a subject exposed to a vesicant. The vesicant is, in some embodiments, a mustard compound, while in other embodiments, it is another vesicant; e.g. another vesicant known in the art. As provided herein, ASC are capable of reducing the incidence of hematological symptoms following exposure to vesicants, as well as prophylactically ameliorating long-term effects of exposure to vesicants.

In some embodiments, there is provided a method of reducing gastrointestinal symptoms of exposure to a vesicant, such as abdominal pain, diarrhea (e.g. bloody diarrhea), hematemesis, nausea and vomiting, comprising the step of administering to the subject a pharmaceutical composition comprising ASC, thereby reducing gastrointestinal symptoms in a subject exposed to a vesicant. The vesicant is, in some embodiments, a mustard compound, while in other embodiments, it is another vesicant; e.g. another vesicant known in the art. As provided herein, ASC are capable of reducing the incidence of gastrointestinal symptoms following exposure to vesicants, as well as prophylactically ameliorating long-term effects of exposure to vesicants.

In some embodiments, there is provided a method of reducing chronic dermal symptoms of exposure to a vesicant, such as dermal pathologies mentioned herein, comprising the step of administering to the subject a pharmaceutical composition comprising ASC, thereby reducing chronic dermal symptoms in a subject exposed to a vesicant. The vesicant is, in some embodiments, a mustard compound, while in other embodiments, it is another vesicant; e.g. another vesicant known in the art. As provided herein, ASC are capable of reducing the incidence of dermal symptoms following exposure to vesicants, as well as prophylactically ameliorating long-term effects of exposure to vesicants.

In some embodiments, there is provided a method of reducing chronic ocular symptoms of exposure to a vesicant, such as ocular pathologies mentioned herein, comprising the step of administering to the subject a pharmaceutical composition comprising ASC, thereby reducing chronic ocular symptoms in a subject exposed to a vesicant. The vesicant is, in some embodiments, a mustard compound, while in other embodiments, it is another vesicant; e.g. another vesicant known in the art. As provided herein, ASC are capable of reducing the incidence of ocular symptoms following exposure to vesicants, as well as prophylactically ameliorating long-term effects of exposure to vesicants.

In still other embodiments, there is provided a method of reducing central nervous system (CNS) damage in a subject exposed to a cholinesterase inhibitor, comprising the step of administering to the subject a pharmaceutical composition comprising ASC, thereby reducing CNS damage in a subject exposed to a cholinesterase inhibitor. As provided herein, ASC are capable of reducing the incidence of CNS damage following exposure to cholinesterase inhibitors, as well as prophylactically ameliorating long-term effects of exposure to cholinesterase inhibitors.

In certain embodiments, the cholinesterase inhibitor is an acetylcholinesterase inhibitor, for example an organophosphorus agent, or in other embodiments, a carbamate.

In certain embodiments, the CNS damage may result from CNS inflammation. Alternatively or in addition, the CNS damage comprises learning deficits, memory impairment, insomnia, or a personality alteration, each of which represents a separate embodiment. In other embodiments, CNS damage includes neurodegeneration. Methods for assessing neurodegeneration following exposure to nerve agents and other cholinesterase inhibitors are known in the art, and include, for example, those described in Grauer E et al, Katalan S et al, Chapman S et al, and the references cited therein.

In yet other embodiments, there is provided a method of reducing muscle damage in a subject exposed to a cholinesterase inhibitor, comprising the step of administering to the subject a pharmaceutical composition comprising ASC, thereby reducing muscle damage in a subject exposed to a cholinesterase inhibitor. In certain embodiments, the muscle damage results from OPIDP syndrome (OP poisoning induced delayed polyneuropathy). As provided herein, ASC are capable of reducing the incidence of muscle damage following exposure to cholinesterase inhibitors. In certain embodiments, the cholinesterase inhibitor is an organophosphorus agent, or in other embodiments, a carbamate.

In still other embodiments, there is provided a method of preventing, or alleviating development of, brain damage in a subject exposed to a cholinesterase inhibitor, by administration of ASC, or in other embodiments CM derived from the ASC. Methods for assessing brain damage following exposure to organophosphorus compounds are known in the art, and include, for example, those described in Finkelstein A et al, Golderman V et al, Rosman Y et al, Shrot S et al, and the references cited therein. In certain embodiments, the cholinesterase inhibitor is an organophosphorus agent, or in other embodiments, a carbamate.

In other embodiments, there is provided a method of reducing respiratory damage in a subject exposed to a pulmonary agent, by administration of ASC, or in other embodiments CM derived from the ASC. Pulmonary agents may also be referred to as choking agents. Methods for assessing respiratory damage following exposure to pulmonary agents are known in the art, and include, for example, those described in Rivkin I et al, 2016, Patel B V et al, Massa C B et al, and the references cited therein. In some embodiments, the respiratory damage is chronic damage, e.g. damage occurring in survivors of acute respiratory distress immediately following exposure. In more specific embodiments, the respiratory damage is manifest as pneumonitis, pulmonary edema, and/or reactive airway disease. In still other embodiments, the respiratory damage is caused at least in part by oxidative stress. Pulmonary agents are known in the art, and are described, for example in McElroy and Day.

Also provided herein are allogeneic placental ASCs for use in a method of mitigating damage or morbidity following exposure to a vesicant, said method comprising the steps of: (a) administering a first pharmaceutical composition, comprising allogeneic placental ASC from a first donor; and subsequently (b) administering a second pharmaceutical composition comprising allogeneic placental ASC from a second donor, wherein the second donor differs from the first donor in at least one allele group of HLA-A or HLA-B; wherein the administrations are separated in time from each other by at least 7 days. In various embodiments, the ASC may be maternal, fetal, or a mixture thereof.

Also provided herein are allogeneic placental ASCs for use in a method of mitigating damage or morbidity following exposure to a cholinesterase inhibitor, said method comprising the steps of: (a) administering a first pharmaceutical composition, comprising allogeneic placental ASC from a first donor; and subsequently (b) administering a second pharmaceutical composition comprising allogeneic placental ASC from a second donor, wherein the second donor differs from the first donor in at least one allele group of HLA-A or HLA-B; wherein the administrations are separated in time from each other by at least 7 days. In various embodiments, the ASC may be maternal, fetal, or a mixture thereof.

Also provided herein are allogeneic placental ASCs for use in a method of mitigating damage or morbidity following exposure to a chemical agent, said method comprising the steps of: (a) administering a first pharmaceutical composition, comprising allogeneic placental ASC from a first donor; and subsequently (b) administering a second pharmaceutical composition comprising allogeneic placental ASC from a second donor, wherein the second donor differs from the first donor in at least one allele group of HLA-A or HLA-B; wherein the administrations are separated in time from each other by at least 7 days. In various embodiments, the ASC may be maternal, fetal, or a mixture thereof.

In certain embodiments, the described first ASC population and second ASC population are derived from the same tissue, which may be, in some embodiments, adipose tissue, or, in other embodiments, bone marrow. In still other embodiments, the tissue is another source of ASC.

Allogeneic, as used herein (except where indicated otherwise), refers to a biological material (e.g. ASC) not derived from, and not syngeneic with, the subject being treated. Typically, allogeneic ASC are neither syngeneic nor haploidentical with the subject.

In certain embodiments, the described allogeneic ASC from the first donor and the second donor (also referred to herein as “first ASC population” and “second ASC population”, respectively) are derived from the same tissue, which may be, in some embodiments, placenta. In other embodiments, the tissue is adipose, or is bone marrow. In still other embodiments, the tissue is another source of ASC.

Alternatively or in addition, the first ASC population and second ASC population exhibit common characteristics. In some embodiments, the common characteristics relate to the cells' therapeutic potential. Certain embodiments of such common characteristics are described herein. In other embodiments, the common characteristic is selected from population doubling time (PDL; this parameter may be derived from population doubling level) and glucose consumption rate (GCR), or in other embodiments is a combination thereof. In certain embodiments, the PDL and/or GCR are measured in bioreactor culture in 3D fibrous carriers, e.g. as described herein in Example 4, following cell expansion as described in Example 1, or in other embodiments, in Examples 2-3. In certain embodiments, the 2 populations are within 2 fold of each other for GCR on day 5 of bioreactor culture. In other embodiments, the GCR is measured on day 3, day 4, or day 6. Alternatively or in addition, the 2 populations are within 1.5 fold, within 3 fold, or within 5 fold of each other for the specified parameter.

Reference to ASC “from” or “derived from” a donor is intended to encompass cells removed from or otherwise obtained from the donor, followed by optional steps of ex-vivo cell culture, expansion, and/or other treatments to improve the therapeutic efficacy of the cells; and/or combination with pharmaceutical excipients. Those skilled in the art will appreciate that the aforementioned optional steps will not alter the HLA genotype of the ASC, absent intentional modification of the HLA genotype (e.g. using CRISPR-mediating editing or the like). Cell populations with an intentionally modified HLA genotype are not intended to be encompassed. ASC populations that contain a mixture cells from more than one donor are also not intended to be encompassed.

As will be appreciated by those skilled in the art, the HLA system or complex is a gene complex encoding the major histocompatibility complex (MHC) proteins in humans. These cell-surface proteins are involved in regulation of the immune system in humans. The HLA gene complex resides on a 3-Mbp stretch within chromosome 6p21. HLA genes are highly polymorphic. HLAs encoding MHC class I proteins (“class I HLA's”) present peptides from inside the cell, while class I HLA's present external peptides.

There are 3 major MHC class I genes, HLA-A, HLA-B, and HLA-C; and 3 minor class I genes, HLA-E, HLA-F and HLA-G. β2-microglobulin binds with major and minor gene subunits to produce a heterodimer.

There are 3 major (DP, DQ and DR) and 2 minor (DM and DO) MHC class II proteins encoded by the HLA. The class II MHC proteins combine to form heterodimeric (αβ) protein receptors that are typically expressed on the surface of antigen-presenting cells.

HLA alleles are often named according to a multi-partite system, where the letter prefix (e.g. “HLA-A”) denotes the locus, followed by an asterisk, followed by the “allele group” number, followed by the specific HLA protein number, followed by a number used to denote silent DNA mutations in a coding region, followed by, lastly, a number used to denote DNA mutations in a non-coding region (Robinson J et al). Typically, the allele group corresponds to the encoded serological antigen, while specific HLA proteins within an allele group exhibit relatively minor antigenic differences. For example, in the hypothetical allele “HLA-A*02:07:01:03”, the allele group number is 02; 07 is the specific HLA protein number, 01 describes a pattern of silent DNA mutations in the coding regions; and 03 describes a pattern of DNA mutations in non-coding regions. “Mutations” in this regard refers to variations relative to the founder (initially identified) allele in the allele group.

HLA typing at each locus, may be, in some embodiments, low resolution, or “first-level field” typing, by reference to the two digits preceding the first separator, or antigen level typing, e.g. A*02 in the above example. In various other embodiments, the typing is at “intermediate-level”resolution, i.e. second-level field, e.g. HLA-A*02:07, or in other embodiments, third-level field, e.g. HLA-A*02:07:01. In other embodiments, the typing is “allele level typing”, using all digits in the first, second, third and fourth fields, e.g. HLA-A*02:07:01:03.

Allele groups are clustered into “supertypes” which have similar peptide binding repertoires. Examples of HLA-A supertypes are 1, 2, 3, and 24, and examples of HLA-B supertypes are 7, 27, 44, 58, and 62.

Reference to a second donor “differ/differs/differing” from a first donor in at least one allele group of HLA-A or HLA-B denotes that the DNA of the second donor comprises at least one HLA-A or HLA-B allele belonging to an allele group not represented in the alleles of the first donor. (Typically [except in the case of a homozygous first donor], the DNA of the first donor will also comprise at least one HLA-A or HLA-B allele belonging to an allele group not represented in the alleles of the second donor). Similarly, a second donor “differs from” a first donor in at least one allele supertype if the DNA of the second donor comprises at least one HLA-A or HLA-B allele belonging to a supertype not represented in the alleles of the first donor. These terms are intended to be used analogously in various contexts herein, except where indicated otherwise.

In other embodiments, the second donor in the described therapeutic methods and compositions differs from the first donor in at least one allele group of HLA-A. In still other embodiments, the second donor differs from the first donor in at least one allele group of HLA-B.

In yet other embodiments, the second donor differs from the first donor in at least two HLA-A allele groups of or, in other embodiments, in at least 2 HLA-B allele groups; or, in other embodiments, at least one allele group of each of HLA-A and HLA-B.

In other embodiments, the second donor differs from the first donor in at least one HLA-A allele supertype or, in other embodiments, at least one HLA-B allele supertype.

In still other embodiments, the second donor differs from the first donor in at least two allele supertypes of HLA-A or HLA-B, which may be, in more specific embodiments, an HLA-A allele supertype, an HLA-B allele supertype, or a combination thereof.

Alternatively or in addition, the second donor differs from the first donor in at least one allele group of HLA-DR, or in other embodiments, in 2 HLA-DR allele groups.

Step b) of the described method (administering a second pharmaceutical composition comprising allogeneic ASC from a second donor) is, in various embodiments, performed between 2-52 weeks. In other embodiments, step b) is performed between 3-52, 4-26, 5-26, 6-20, 6-18, 6-15, 6-10, 3-20, 3-15, 3-10, 4-12, 4-20, 5-18, 6-16, 8-16, 10-16, or 8-12 weeks after step a).

Alternatively or in addition, step b) of the described methods is followed by an additional step, comprising the step of administering to the subject, at least 7 days after step b), a third pharmaceutical composition comprising allogeneic ASC derived from a third donor, wherein the third donor differs from both the first donor and the second donor in at least one allele group of HLA-A or HLA-B (i.e. has an allele group not represented in either the first or second donor), which is, in various embodiments, an allele of HLA-A or HLA-B. In other embodiments, the third donor differs from both the first donor and the second donor in at least two allele groups of HLA-A or HLA-B, which are, in various embodiments, an allele of HLA-A, HLA-B, or a combination thereof.

In certain embodiments, the ASC are derived from placenta, from adipose tissue, or from BM.

“Vesicant” refers to a chemical that blisters the exposed tissue of a subject following contact. In certain embodiments, the eyes, respiratory tract, and/or skin of a subject are affected. Non-limiting examples of vesicants are Lewisite (chlorovinylarsine dichloride); mustard compounds, e.g. sulfur mustard (bis(2-chloroethyl)sulfide), nitrogen mustard HN-1 (bis(2-chloroethyl)ethylamine), nitrogen mustard HN-2 (bis(2-chloroethyl)methylamine), nitrogen mustard HN-3 (tris(2-chloroethyl)amine), 2-chloroethyl ethylsulfide (2-CEES), agent HT (a mixture of distilled mustard and Agent T [Bis(2-chloroethylthioethyl) ether]), and a mustard-Lewisite mixture (e.g. a sulfur mustard-Lewisite mixture); phosgene oxime (dichloroformoxime); and cantharidin (2,6-dimethyl-4,10-dioxatricyclo-[5.2.1.02,6]decane-3,5-dione). In other embodiments, the vesicant is an organic arsenic compound, e.g. ethyldichloroarsine (ethylarsonous dichloride), methyldichloroarsine (dichloromethylarsane), and phenyldichloroarsine (phenylarsonous dichloride). In certain embodiments, the vesicant is an arsenical vesicant compound (described under the heading Arsenic Compounds in the Toxnet database of the US National Library of Medicine, accessed on Feb. 22, 2017). As will be appreciated by those skilled in the art, both distilled and undistilled chemical agents can cause vesication.

Non-limiting examples of organophosphorus agents include phosphate esters (having the general structure P(═O)(OR)₃) and amides (also including derivatives containing the thiophosphoryl group (P═S), e.g. malathion); phosphonic acids (having the general formula RP(═O)(OR′)₂; e.g. sarin, soman, and GF) and esters, e.g. phosphonothiolates such as VX; phosphinic acids (having the general formula R₂P(═O)(OR′)) and esters thereof; and phosphine oxides (having the general structure R₃P═O with formal oxidation state V). Other examples of organophosphorus agents include phosphites, phosphonites, phosphinites, and phosphines. Still other embodiments of organophosphorus agents are nerve agents, such as G agents, e.g. GA (tabun), GB (sarin; (RS)-propan-2-yl methylphosphonofluoridate), GD (soman), and GF (cyclosarin); V agents, e.g. EA-3148 (O-cyclopentyl S-(2-diethylaminoethyl) methylphosphonothiolate), VE, VG, VM, VR (Russian V-gas or N,N-diethyl-2-(methyl-(2-methylpropoxy)phosphoryl) sulfanylethanamine), VP ((3,3,5-Trimethylcyclohexyl-3-pyridylmethylphosphonate), and VX (O-ethyl S-diisopropylaminomethyl methylphosphonothiolate). Yet other embodiments of organophosphorus agents are pesticides and insecticides, non-limiting examples of which are dichlorvos, malathion, paraoxon (diethyl 4-nitrophenyl phosphate), and parathion. Domestic exposure to pesticides may be complicated by exposure to (e.g. ingestion of) organic solvents in which the pesticides are dissolved or suspended.

Non-limiting examples of carbamates include physostigmine, neostigmine, pyridostigmine, ambenonium, and demecarium.

Non-limiting examples of pulmonary agents include chlorine; chloropicrin (trichloro(nitro)methane); phosgene (carbonyl dichloride); phosphine; diphosgene; and disulfur decafluoride. The pulmonary agent is, in various embodiments, a central pulmonary agent (primarily causing damage to airways larger than 2 millimeters [mm]; e.g. chlorine), or a peripheral pulmonary agent (primarily causing damage to airways smaller than 2 mm or alveoli; e.g. phosgene). In certain embodiments, the pulmonary agent is a substance that forms hydrochloric acid and/or hypochlorous acid upon contact with lung epithelial lining fluid.

Reducing morbidity, as used herein, includes, in some embodiments, long-term complications of exposure to chemical agents. In certain embodiments, the subject has been given an antidote to ameliorate short-term effects of the agent, prior to administration of the described composition. For example, BAL (e.g. British-Anti-Lewisite or dimercaprol) is routinely administered for Lewisite. Subjects exposed to sarin or other nerve agents are typically administered atropine, benzodiazepine, and/or an oxime, such as pralidoxime.

As mentioned, the described methods and compositions in some embodiments reduce an incidence of mortality resulting from the described vesicants and chemical agents. In certain embodiments, the mortality results from sepsis, infection, pulmonary damage and/or any combination thereof, each of which is considered a separate embodiment.

As will be appreciated by those skilled in the art, exposure to vesicants and other chemical agents causes detectable respiratory, dermal, cardiovascular, gastrointestinal, central nervous system (CNS), and/or hematological symptoms. In some embodiments, the morbidity treated by described methods and compositions is a respiratory morbidity, a dermal morbidity, a cardiovascular morbidity, a gastrointestinal morbidity, a CNS morbidity, or a hematological morbidity.

Exposure to vesicants, nerve agents, and the like may be assessed by point-of-care medical personnel. Depending on the agents and the dose, one or more of the following symptoms are presented:

Subjects exposed to nerve agents may exhibit ocular pain, darkness of visual field, nausea, vomiting, headache, rhinorrhea, narrowing of visual field, and/or dyspnea. In certain cases, subject exhibit atrioventricular block and cardiac arrest (with high-dose exposure). Long-term symptoms include CNS damage, which may manifest as ataxia, epilepsy, learning difficulties, memory and sleep disturbances. In some embodiments, CNS inflammation is mediated by injury to astrocytes and microglia.

Subjects exposed to pulmonary agents may exhibit acute lung injury. Long-term symptoms include other lung damage, which may be, in non-limiting embodiments, mediated by progressive alteration in surfactant composition and mechanical dysfunction (e.g. from chlorine agents; Massa C B et al).

Subjects exposed to vesicants may exhibit skin blisters (within 1 hour with phosgene oxime, delayed for 2-12 hours with Lewisite, delayed for 2-24 hours with mustards), erythema (immediate with Lewisite and phosgene oxime; may be delayed for 2-24 hours with mustards) immediate blanching (phosgene oxime), itching, and necrosis and eschar (over a period of 7-10 days). Long-term effects of vesicants include pulmonary manifestations such as chronic inflammation, pulmonary fibrosis, pulmonary hypertension, and bronchiolitis obliterans; gastrointestinal manifestations (e.g. following ingestion), such as abdominal pain, diarrhea (e.g. bloody diarrhea), hematemesis, nausea and vomiting; and hematological manifestations, e.g. anemia, bleeding/hemorrhage, bone marrow suppression, increased susceptibility to infection, leukocytopenia, and thrombocytopenia.

Treatment of each of the described symptoms represents a separate embodiment of the present invention.

In various embodiments, the ASC are administered to the subject within 1 hour, within 2 hours, within 3 hours, within 4 hours, within 6 hours, within 8 hours, within 10 hours, within 12 hours, within 15 hours, within 18 hours, within 24 hours, within 30 hours, within 36 hours, within 48 hours, within 3 days, within 4 days, within 5 days, within 6 days, within 8 days, within 10 days, within 12 days, or within 20 days of the exposure. In some embodiments, the described compositions are administered after the subject is stabilized from acute pathologies. In some embodiments, the subject is stabilized by supportive medical care and/or the administration of antidotes. In other embodiments, the described compositions are administered to alleviate long-term damage incurred from the agent. In more specific embodiments, the described compositions are administered 1-24, 2-24, 3-24, 4-24, 5-24, 6-24, 8-24, 10-24, 12-48, 1-48, 2-48, 3-48, 4-48, 5-48, 6-48, 8-48, 10-48, 12-48, 18-48, 24-48, 1-72, 2-72, 3-72, 4-72, 5-72, 6-72, 8-72, 10-72, 12-72, 18-72, 24-72, or 36-72 hours after the exposure (or after the estimated time of the exposure, if the exact time is not known). In still other embodiments, the described compositions are administered 3-48, 4-48, 5-48, or 6-48 hours after the exposure (or after the estimated time of the exposure, if the exact time is not known), to alleviate damage from the agent.

In certain embodiments, any of the described compositions further comprises a pharmacologically acceptable excipient. In further embodiments, the excipient is an osmoprotectant or cryoprotectant, an agent that protects cells from the damaging effect of freezing and ice formation, which may in some embodiments be a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and carrier protein are present; in certain embodiments, the osmoprotectant and carrier protein may be the same compound. Alternatively or in addition, the composition is frozen. The cells may be any embodiment of ASC mentioned herein, each of which is considered a separate embodiment.

In various embodiments, the described cells are able to exert the described therapeutic effects, each of which is considered a separate embodiment, with or without the cells themselves engrafting in the host. For example, the cells may, in various embodiments, be able to exert a therapeutic effect, without themselves surviving for more than 3 days, more than 4 days, more than 5 days, more than 6 days, more than 7 days, more than 8 days, more than 9 days, more than 10 days, or more than 14 days.

ASC and Sources Thereof

In certain embodiments, the described ASC are mesenchymal stromal cells (MSC). These cells may, in some embodiments, be isolated from bone marrow. In further embodiments, the cells are human MSC as defined by The Mesenchymal and Tissue Stem Cell Committee of the International Society for Cellular Therapy (Dominici et al, 2006), based on the following 3 criteria: 1. Plastic-adherence when maintained in standard culture conditions (a minimal essential medium plus 20% fetal bovine serum (FBS)). 2. Expression of the surface molecules CD105, CD73 and CD90, and lack of expression of CD45, CD34, CD14 or CD11b, CD79α, or CD19 and HLA-DR. 3. Ability to differentiate into osteoblasts, adipocytes and chondroblasts in vitro.

In other embodiments, the described ASC are placenta-derived. Except where indicated otherwise herein, the terms “placenta”, “placental tissue”, and the like refer to any portion of the placenta. Placenta-derived adherent cells may be obtained, in various embodiments, from either fetal or, in other embodiments, maternal regions of the placenta, or in other embodiments, from both regions. More specific embodiments of maternal sources are the decidua basalis and the decidua parietalis. More specific embodiments of fetal sources are the amnion, the chorion, and the villi. In certain embodiments, tissue specimens are washed in a physiological buffer [e.g., phosphate-buffered saline (PBS) or Hank's buffer]. In certain embodiments, the placental tissue from which cells are harvested includes at least one of the chorionic and decidua regions of the placenta, or, in still other embodiments, both the chorionic and decidua regions of the placenta.

More specific embodiments of chorionic regions are chorionic mesenchymal and chorionic trophoblastic tissue. More specific embodiments of decidua are decidua basalis, decidua capsularis, and decidua parietalis.

Single-cell suspensions can be made, in other embodiments, by treating the tissue with a digestive enzyme (see below) or/and physical disruption, a non-limiting example of which is mincing and flushing the tissue parts through a nylon filter or by gentle pipetting (e.g. Falcon, Becton, Dickinson, San Jose, Calif.) with washing medium. In some embodiments, the tissue treatment includes use of a DNAse, a non-limiting example of which is Benzonase from Merck.

Placental cells may be obtained, in various embodiments, from a full-term or pre-term placenta. In some embodiments, the placental tissue is optionally minced, followed by enzymatic digestion. Optionally, residual blood is removed from the placenta before cell harvest. This may be done by a variety of methods known to those skilled in the art, for example by perfusion. The term “perfuse” or “perfusion” as used herein refers to the act of pouring or passaging a fluid over or through an organ or tissue. In certain embodiments, the placental tissue may be from any mammal, while in other embodiments, the placental tissue is human. A convenient source of placental tissue is a post-partum placenta (e.g., less than 10 hours after birth), however, a variety of sources of placental tissue or cells may be contemplated by the skilled person. In other embodiments, the placenta is used within 8 hours, within 6 hours, within 5 hours, within 4 hours, within 3 hours, within 2 hours, or within 1 hour of birth. In certain embodiments, the placenta is kept chilled prior to harvest of the cells. In other embodiments, prepartum placental tissue is used. Such tissue may be obtained, for example, from a chorionic villus sampling or by other methods known in the art. Once placental cells are obtained, they are, in certain embodiments, allowed to adhere to an adherent material (e.g., configured as a surface) to thereby isolate adherent cells. In some embodiments, the donor is 35 years old or younger, while in other embodiments, the donor may be any woman of childbearing age.

ASC (e.g. placenta-derived ASC) can be propagated, in some embodiments, by using a combination of 2D and 3D culturing conditions. Conditions for propagating adherent cells in 2D and 3D culture are further described herein below and in the Examples section which follows. Those skilled in the art will appreciate, in light of the present disclosure, that cells may be, in some embodiments, extracted from a placenta, for example using physical and/or enzymatic tissue disruption, followed by marker-based cell sorting, and then may be subjected to the culturing methods described herein.

In further embodiments, the described population of ASC (e.g. placental ASC) expresses one or more markers that are not expressed (or at least not highly expressed) in BM-MSC. In certain embodiments, the expressed markers are selected from any combination of CD46 (Uniprot Acc. No. P15529), CD59 (No. P15529), CD61 (P05106), CD140b (P09619), CD144 (P33151), and CD150 (Q13291). Alternatively or in addition, the cells do not express one or more markers that are expressed in BM-MSC. In certain embodiments, the non-expressed markers are selected from any combination of CD62P (No. P16109), CD109 (Q6YHK3), CD112 (Q92692), and CD154 (P29965). In yet other embodiments, the cells do not express CD9 (No. P21926) at high levels; and/or do express CD55 (P21926) at high levels. See Winkler T et al. Uniprot entries were accessed on Jun. 10, 2019.

In still other embodiments, the cells are a placental cell population that is a mixture of fetal-derived placental ASC (also referred to herein as “fetal ASC” or “fetal cells”) and maternal-derived placental ASC (also referred to herein as “maternal ASC” or “maternal cells”) and contains predominantly maternal cells. In more specific embodiments, the mixture contains at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, at least 99.92%, at least 99.95%, at least 99.96%, at least 99.97%, at least 99.98%, or at least 99.99% maternal cells, or contains between 90-99%, 91-99%, 92-99%, 93-99%, 94-99%, 95-99%, 96-99%, 97-99%, 98-99%, 90-99.5%, 91-99.5%, 92-99.5%, 93-99.5%, 94-99.5%, 95-99.5%, 96-99.5%, 97-99.5%, 98-99.5%, 90-99.9%, 91-99.9%, 92-99.9%, 93-99.9%, 94-99.9%, 95-99.9%, 96-99.9%, 97-99.9%, 98-99.9%, 99-99.9%, 99.2-99.9%, 99.5-99.9%, 99.6-99.9%, 99.7-99.9%, or 99.8-99.9% maternal cells.

Predominantly or completely maternal cell preparations may be obtained by methods known to those skilled in the art, including the protocol detailed in Example 1 and the protocols detailed in PCT Publ. Nos. WO 2007/108003, WO 2009/037690, WO 2009/144720, WO 2010/026575, WO 2011/064669, and WO 2011/132087. The contents of each of these publications are incorporated herein by reference. Predominantly or completely fetal cell preparations may be obtained by methods known to those skilled in the art, including selecting fetal cells via their markers (e.g. a Y chromosome in the case of a male fetus), and expanding the cells.

In other embodiments, the cells are a placental cell population that does not contain a detectable amount of maternal cells and is thus entirely fetal cells. A detectable amount refers to an amount of cells detectable by FACS, using markers or combinations of markers present on maternal cells but not fetal cells, as described herein. In certain embodiments, “a detectable amount” may refer to at least 0.1%, at least 0.2%, at least 0.3%, at least 0.4%, at least 0.5%, at least 0.6%, at least 0.7%, at least 0.8%, at least 0.9%, or at least 1%.

In still other embodiments, the preparation is a placental cell population that is a mixture of fetal and maternal cells and is enriched for fetal cells. In more specific embodiments, the mixture contains at least 70% fetal cells. In more specific embodiments, at least about 30%, 35%, 40%, 45%, 50%, 55%, 60%, 65%, 75%, 80%, 85%, 90%, 95%, 96%, 97%, 98%, 99% or 100% of the cells are fetal cells. Expression of CD200, as measured by flow cytometry, using an isotype control to define negative expression, can be used as a marker of fetal cells under some conditions. In yet other embodiments, at least 60%, at least 65%, at least 70%, at least 75%, at least 80%, at least 85%, at least 90%, at least 92%, at least 95%, at least 96%, at least 97%, at least 98%, at least 99%, at least 99.5%, at least 99.7%, or at least 99.9% of the described cells are fetal cells.

In more specific embodiments, the mixture contains 20-80% fetal cells; 30-80% fetal cells; 40-80% fetal cells; 50-80% fetal cells; 60-80% fetal cells; 20-90% fetal cells; 30-90% fetal cells; 40-90% fetal cells; 50-90% fetal cells; 60-90% fetal cells; 20-80% maternal cells; 30-80% maternal cells; 40-80% maternal cells; 50-80% maternal cells; 60-80% maternal cells; 20-90% maternal cells; 30-90% maternal cells; 40-90% maternal cells; 50-90% maternal cells; or 60-90% maternal cells.

As used herein, the phrase “adipose tissue” refers to a connective tissue which comprises fat cells (adipocytes). Adipose tissue-derived adherent stromal cells may be extracted, in various embodiments, by a variety of methods known to those skilled in the art, for example those described in U.S. Pat. No. 6,153,432, which is incorporated herein by reference. The adipose tissue may be derived, in other embodiments, from omental/visceral, mammary, gonadal, or other adipose tissue sites. In some embodiments, the adipose can be isolated by liposuction.

In other embodiments, ASC may be derived from adipose tissue by treating the tissue with a digestive enzyme (non-limiting examples of which are collagenase, trypsin, dispase, hyaluronidase or DNAse); and ethylenediaminetetra-acetic acid (EDTA). The cells may be, in some embodiments, subjected to physical disruption, for example using a nylon or cheesecloth mesh filter. In other embodiments, the cells are subjected to differential centrifugation directly in media or over a Ficoll or Percoll or other particulate gradient (see U.S. Pat. No. 7,078,230, which is incorporated herein by reference).

In still other embodiments, the ASC are derived from peripheral blood; umbilical cord blood; synovial fluid; synovial membranes; spleen; thymus; mucosa (for example nasal mucosa); limbal stroma; ligament (e.g. periodontal ligament); dermis; scalp; hair follicles, testicles; embryonic yolk sac; muscle tissue; or amniotic fluid. In some embodiments, the ASC are human ASC, while in other embodiments, they may be animal ASC. In still other embodiments, they are allogeneic human ASC.

In another embodiment, the described cell population is produced by expanding a population of ASC in a medium that contains less than 5% animal serum.

In certain embodiments, the aforementioned medium contains less than 4% animal serum; less than 3% animal serum; less than 2% animal serum; less than 1% animal serum; less than 0.5% animal serum; less than 0.3% animal serum; less than 0.2% animal serum; or less than 0.1% animal serum. In other embodiments, the medium does not contain animal serum. In other embodiments, the medium is a defined medium to which no serum has been added. Low-serum and serum-free media are collectively referred to as “serum-deficient medium/media”.

Those skilled in the art will appreciate that reference herein to animal serum includes serum from a variety of species, provided that the serum stimulates expansion of the ASC population. In certain embodiments, the serum is mammalian serum, non-limiting examples of which are human serum, bovine serum (e.g. fetal bovine serum and calf bovine serum), equine serum, goat serum, and porcine serum.

In certain embodiments, the serum-deficient medium is supplemented with factors intended to stimulate cell expansion in the absence of serum. Such medium is referred to herein as serum-replacement medium or SRM, and its use, for example in cell culture and expansion, is known in the art, and is described, for example, in Kinzebach et al.

In other embodiments, the serum-deficient medium contains one or more growth factors. In certain embodiments, the growth factors, individually or in other embodiments collectively, induce cell expansion in culture. In other embodiments, the growth factors, individually or, in other embodiments collectively, induce cell expansion in culture without differentiation.

In more specific embodiments, the factor(s) contained in the serum-deficient medium is selected from a FGF, TGF-beta (Uniprot accession no. P01137), transferrin (e.g. serotransferrin or lactotransferrin; Uniprot accession nos. P02787 and P02788), insulin (Uniprot accession no. P01308), EGF (epidermal growth factor; Uniprot accession no. P01133), and/or PDGF (platelet-derived growth factor, including any combination of subunits A and B; Uniprot accession nos. P04085 and P01127), each of which represents a separate embodiment. A non-limiting example of PDGF is PDGF-BB.

Except where indicated otherwise, reference herein to a protein includes all its isoforms functional fragments thereof, and mimetics thereof. Such reference also includes homologues from a variety of species, provided that the protein acts on the target cells in a similar fashion to the homologue from the same species as the target cells. For example, if human cells are being expanded, reference to bFGF would also include any non-human bFGF that stimulates proliferation of human cells. Those skilled in the art will appreciate that, even in the case of human cells, the aforementioned proteins need not be human proteins, since many non-human (e.g. animal) proteins are active on human cells. Similarly, the use of modified proteins that have similar activity to the native forms falls within the scope of the described methods and compositions.

The FGF (fibroblast growth factor) family includes a number of proteins that are described in Imamura. A non-limiting example is bFGF (Uniprot accession no. P09038).

In other embodiments, the serum-deficient medium comprises an FGF and TGF-beta. In still other embodiments, the medium comprises an FGF, TGF-beta, and PDGF. In more specific embodiments, the medium further comprises transferrin, insulin, or both transferrin and insulin. Alternatively or in addition, the medium further comprises oleic acid.

In still other embodiments, the serum-deficient medium comprises an FGF and EGF. In still other embodiments, the medium further comprises transferrin, insulin, or both transferrin and insulin.

SRM formulations include MSC Nutristem® XF (Biological Industries); Stempro® SFM and Stempro® SFM-XF (Thermo Fisher Scientific); PPRF-msc6; D-hESF10; TheraPEAK™ MSCGM-CD™ (Lonza, cat. no. 190632); and MesenCult-XF (Stem Cell Technologies, cat. no. 5429). The StemPro® media contain PDGF-BB, bFGF, and TGF-P, and insulin (Chase et al). The composition of PPRF-msc6 is described in US 2010/0015710, which is incorporated herein by reference. D-hESF10 contains insulin (10 micrograms per milliliter [mcg/ml]); transferrin (5 mcg/ml); oleic acid conjugated with bovine albumin (9.4 mcg/ml); FGF-2 (10 ng/ml); and TGF-β1 (5 ng/ml), as well as heparin (1 mg/ml) and standard medium components (Mimura et al).

As provided herein, ASC were expanded in Stempro® SFM-XF. MSC Nutristem® XF was also tested and yielded similar results. Additionally, an in-house medium was produced and tested, containing DMEM/F-12 supplemented with 50 ng/ml PDGF-BB, 15 ng/ml bFGF, and 2 ng/ml TGF-P. This medium yielded similar results to Stempro® SFM-XF. DMEM/F-12 is a known basal medium, available commercially from Thermo Fisher Scientific (cat. no. 10565018).

Another SRM formulation is described in Rajaraman G et al and contains FGF-2 (10 ng/ml); epidermal growth factor (EGF) (10 ng/ml); 0.5% BSA; Insulin (10 mcg/ml); transferrin (5.5 mcg/ml); 6.7 ng/mL sodium selenite, sodium pyruvate (11 mcg/ml); heparin (0.1 mg/ml); 10 nM linolenic acid.

In certain embodiments, the described SRM comprises bFGF (basic fibroblast growth factor, also referred to as FGF-2), TGF-β (TGF-β, including all isotypes, for example TGFβ1, TGFβ2, and TGFβ3), or a combination thereof. In other embodiments, the SRM comprises bFGF, TGF-β, and PDGF. In still other embodiments, the SRM comprises bFGF and TGF-β, and lacks PDGF-BB. Alternatively or in addition, insulin is also present. In still other embodiments, an additional component selected from ascorbic acid, hydrocortisone and fetuin is present; 2 components selected from ascorbic acid, hydrocortisone and fetuin are present; or ascorbic acid, hydrocortisone and fetuin are all present.

In other embodiments, the described SRM comprises bFGF, TGF-β, and insulin. In additional embodiments, a component selected from transferrin (5 mcg/ml) and oleic acid are present; or both transferrin and oleic acid are present. Oleic acid can be, in some embodiments, conjugated with a protein, a non-limiting example of which is albumin. In some embodiments, the SRM comprises 5-20 ng/ml bFGF, 2-10 ng/ml TGF-β, and 5-20 ng/ml insulin, or, in other embodiments, 7-15 ng/ml bFGF, 3-8 ng/ml TGF-β, and 7-15 ng/ml insulin. In more specific embodiments, a component selected from 2-10 mcg/ml transferrin and 5-20 mcg/ml oleic acid, or in other embodiments, a component selected from 3-8 mcg/ml transferrin and 6-15 mcg/ml oleic acid, or in other embodiments the aforementioned amounts of both components (transferrin and oleic acid) is/are also present.

In yet other embodiments, the described SRM comprises bFGF and EGF. In more specific embodiments, the bFGF and EGF are present at concentrations independently selected from 5-40, 5-30, 5-25, 6-40, 6-30, 6-25, 7-40, 7-30, 7-25, 7-20, 8-, 8-17, 8-15, 8-13, 9-20, 9-17, 9-15, 10-15, 5-20, 5-10, 7-13, 8-12, 9-11, or 10 ng/ml. In certain embodiments, insulin; and/or transferrin is also present. In more specific embodiments, the insulin and transferrin are present at respective concentrations of 5-20 and 2-10; 6-18 and 3-8; or 8-15 and 4-7 mcg/ml. Alternatively or in addition, the SRM further comprises an additional component selected from BSA, selenite (e.g. sodium selenite), pyruvate (e.g. sodium pyruvate); heparin, and linolenic acid. In other embodiments 2 or more, or in other embodiments 3 or more, in other embodiments 4 or more, or in other embodiments all 5 of BSA, selenite, pyruvate, heparin, and linolenic acid are present. In more specific embodiments, the BSA, selenite, pyruvate, heparin, and linolenic acid are present at respective concentrations of 0.1-5%, 2-30 ng/mL, 5-25 mcg/ml, 0.05-0.2 mg/ml, and 5-20 nM; or in other embodiments at respective concentrations of 0.2-2%, 4-10 ng/mL, 7-17 mcg/ml, 0.07-0.15 mg/ml, and 7-15 nM; or in other embodiments the aforementioned amounts or 2 or more, or in other embodiments 3 or more, in other embodiments 4 or more, or in other embodiments all 5 of BSA, selenite, pyruvate, heparin, and linolenic acid are present.

In other embodiments, bFGF, where present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 8-13, 8-12, 9-11, 9-12, about 10, or 10 nanograms per milliliter (ng/ml).

In other embodiments, EGF, where present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 7-25, 7-22, 8-25, 8-22, 9-21, 10-20, 8-13, 8-12, 9-11, 9-12, about 10, or 10 ng/ml.

In other embodiments, TGF-β, where present, is present at a concentration of 1-25, 2-25, 3-25, 4-25, 5-25, 1-20, 1-15, 1-10, 1-8, 1-7, 1-6, 1-5, 2-20, 2-15, 2-10, 3-20, 3-15, 3-10, 3-8, 3-7, 4-8, 4-7, 4-6, 4.5-5.5, about 5, or 5 ng/ml.

In other embodiments, PDGF, where present, is present at a concentration of 1-50, 1-40, 1-30, 1-20, 1-15, 1-10, 1-8, 1-7, 1-6, 1-5, 1-4, 1-3, 1-2, 2-50, 2-40, 2-30, 2-20, 2-15, 2-10, 2-8, 2-7, 2-6, 2-5, 2-4, 3-50, 3-40, 3-30, 3-20, 3-15, 3-10, 3-8, 3-7, 3-6, 3-5, 3-4, 4-40, 4-30, 4-20, 5-40, 5-30, 5-20, 5-15, 5-12, 5-10, 10-20, 10-18, 10-16, or 10-15, 2-20, about 2, about 3, about 5, about 10, about 15, about 20, 2, 3, 5, 10, 15, or 20 ng/mL.

In still other embodiments, the ASC are expanded in a multi-step process, including the steps of (a) incubating a population of ASC in a serum-deficient medium, thereby obtaining a first expanded cell population; and (b) incubating the first expanded cell population in a second medium, wherein the second medium contains at least 10% animal serum.

The aforementioned second medium, in some embodiments, contains an animal serum content of 5-25%, 6-25%, 7-25%, 8-25%, 9-25%, 10-25%, 11-25%, 12-25%, 13-25%, 14-25%, 15-25%, 10-24%, 10-23%, 10-22%, 10-21%, 10-20%, 11-19%, 12-18%, 13-17%, 16-24%, 17-23%, or 18-22%. In other embodiments, the second medium contains at least 11%, 12%, 13%, 14%, 15%, 16%, 17%, 18%, 19%, or 20% animal serum. In certain embodiments, the second medium does not contain added growth factors, other than those present in the animal serum added thereto.

In still other embodiments, the described methods are preceded by an earlier step wherein cells are cultured in serum-containing medium, prior to culturing in a serum-deficient medium. The serum-containing medium can be, in certain embodiments, any standard growth medium. Non-limiting examples, for exemplary purposes only, are DMEM+10% FBS and DMEM+5% human serum. A non-limiting example of these embodiments is use of standard growth medium to incubate and expand cells, for a limited number of passages (e.g. 1-3 passages, or in other embodiments 2-10 doublings) following their extraction from the source tissue, followed by expansion in serum-deficient medium, which is, in some embodiments, in turn followed by further expansion in serum-containing medium. As provided herein, the initial use of serum-containing medium, for example after extraction, facilitates, in some scenarios, initial attachment and expansion of cells after their extraction. In certain embodiments, the earlier step is performed on a 2D substrate.

In some embodiments, the step of incubating an ASC population in serum-deficient medium is performed on a 2D substrate; and at least a portion of the subsequent step (incubating the expanded cell population in a serum-containing medium) is performed on a 3D substrate. In certain embodiments, the 3D substrate is in a bioreactor. Alternatively or in addition, the 3D substrate is a synthetic adherent material. These embodiments of methods may be freely combined with any of the described embodiments of bioreactors, adherent materials, and/or 3D carriers and substrates. In still other embodiments, the aforementioned subsequent step is initiated on a 2D substrate for a duration of at least 2, at least 3, at least 4, at least 5, at least 6, 2-10, 3-10, 4-10, 5-10, 2-8, 3-8, 4-8, or 5-8 cell doublings, before performing additional expansion in a serum-containing medium on a 3D substrate. The 2D substrate on which the subsequent step is initiated may be the same or different from the 2D substrate on which the described earlier step was performed, where applicable.

Other Culture Embodiments

In other embodiments, the placental ASC are cultured in the presence of extracts, or in other embodiments CM, from ischemic cells. Non-limiting example of such protocols are described in Cha et al.

In yet other embodiments, the placental ASC are cultured, or in other embodiments incubated, under hypoxic conditions. Methods for hypoxia preconditioning are known in the art; non-limiting examples of such methods include treatment with 0.1-0.3% 02, treatment with 0.5% 02, e.g. for 24 hours; treatment with a 1% 02 and 5% C02 atmosphere, in some embodiments in glucose-free medium, e.g. for 24 hours; culturing at 2%, 3% or 5% O₂ for 1-7 days; culturing at 5% 02 for 2 days; and culturing in 95% 02. Also encompassed, in other embodiments, are regimens of hypoxia preconditioning (which may be 1-5%, e.g. about 2.5% 02), reoxygenation (e.g. at ambient conditions, which may be 15-25%, e.g. about 21% 02), and further hypoxia preconditioning (which may be 1-5%, e.g. about 2.5% 02); for example as described in Hu C and Li L, Liu J et al, Sun J et al, Boyette L B et al, Kheirandish M et al., Kim Y S et al., Barros S et al, Waszak P et al, and the references cited therein.

In yet other embodiments, the placental ASC are subjected to pharmacological preconditioning, non-limiting examples of which are treatment with Deferoxamine (DFO), polyribocytidylic acid, and other toll-like receptor-3 (TLR3) agonists. Protocols for pharmacological preconditioning are known in the art; non-limiting examples of such methods are described in Najafi R et al, Qiu Y et al, Uu X et al, and Hu C and Li L, and the references cited therein.

In yet other embodiments, the placental ASC are subjected to preconditioning with one or more hormones, non-limiting examples of which are oxytocin, melatonin, all-trans retinoic acid, SDF-1/CXCR4 (Uniprot Accession No. P61073), Oncostatin M (Uniprot Accession No. P13725), and TGF-beta-1 (Uniprot Accession No. P01137), interferon-gamma (Uniprot Accession No. P01579), and migration inhibitory factor (Uniprot Accession No. P14174). Protocols for hormone preconditioning are known in the art; non-limiting examples of such methods are described in Noiseux N et al, Tang Y et al, Pourjafar M et al, Lan Y W et al, Li D et al, Duijvestein M et al, Xia W, Hu C and Li L, and the references cited therein.

In still other embodiments, the placental ASC are subjected to preconditioning with laser light, pulsed electromagnetic fields (PEMF), or nanoparticles and/or microparticles (e.g. silica particles). Protocols for such treatments are known in the art, and non-limiting examples are described in Yin K et al, Urnukhsaikhan E et al, Kim K J et al, and the references cited therein.

In certain embodiments, the preconditioned placental ASC are indicated for treating respiratory distress syndrome. In other embodiments, the described preconditioning methods may be freely combined with other culturing and cell expansion methods described herein. In certain embodiments, the placental cells used for the described hematopoietic indications are fetal cells.

Surface Markers and Additional Characteristics of ASC

Alternatively or additionally, the ASC may express a marker or a collection of markers (e.g. surface marker) characteristic of MSC or mesenchymal-like stromal cells. In some embodiments, the ASC express some or all of the following markers: CD105 (UniProtKB Accession No. P17813), CD29 (UniProtKB Accession No. P05556), CD44 (UniProtKB Accession No. P16070), CD73 (UniProtKB Accession No. P21589), and CD90 (UniProtKB Accession No. P04216). In some embodiments, the ASC do not express some or all of the following markers: CD3 (e.g. UniProtKB Accession Nos. P09693 [gamma chain] P04234 [delta chain], P07766 [epsilon chain], and P20963 [zeta chain]), CD4 (UniProtKB Accession No. P01730), CD11b (UniProtKB Accession No. P11215), CD14 (UniProtKB Accession No. P08571), CD19 (UniProtKB Accession No. P15391), and/or CD34 (UniProtKB Accession No. P28906). In more specific embodiments, the ASC also lack expression of CD5 (UniProtKB Accession No. P06127), CD20 (UniProtKB Accession No. P11836), CD45 (UniProtKB Accession No. P08575), CD79-alpha (UniProtKB Accession No. B5QTD1), CD80 (UniProtKB Accession No. P33681), and/or HLA-DR (e.g. UniProtKB Accession Nos. P04233 [gamma chain], P01903 [alpha chain], and P01911 [beta chain]). The aforementioned, non-limiting marker expression patterns were found in certain maternal placental cell populations that were expanded on 3D substrates. All UniProtKB entries mentioned in this paragraph were accessed on Jul. 7, 2014. Those skilled in the art will appreciate that the presence of complex antigens such as CD3 and HLA-DR may be detected by antibodies recognizing any of their component parts, such as, but not limited to, those described herein.

In some embodiments, the ASC possess a marker phenotype that is distinct from bone marrow-mesenchymal stem cells (BM-MSC). In certain embodiments, the ASC are positive for expression of CD10 (which occurs, in some embodiments, in both maternal and fetal ASC); are positive for expression of CD49d (which occurs, in some embodiments, at least in maternal ASC); are positive for expression of CD54 (which occurs, in some embodiments, in both maternal and fetal ASC); are bimodal, or in other embodiments positive, for expression of CD56 (which occurs, in some embodiments, in maternal ASC); and/or are negative for expression of CD106. Except where indicated otherwise, bimodal refers to a situation where a significant percentage (e.g. at least 20%) of a population of cells express a marker of interest, and a significant percentage do not express the marker.

In certain embodiments, over 90% of the ASC are positive for CD29, CD90, and CD54. In other embodiments, over 85% of the described cells are positive for CD29, CD73, CD90, and CD105. In yet other embodiments, less than 3% of the described cells are positive for CD14, CD19, CD31, CD34, CD39, CD45RA (an isotype of CD45), HLA-DR, Glycophorin A, and CD200; less than 6% of the cells are positive for GlyA; and less than 20% of the cells are positive for SSEA4. In more specific embodiments, over 90% of the described cells are positive for CD29, CD90, and CD54; and over 85% of the cells are positive for CD73 and CD105. In still other embodiments, over 90% of the described cells are positive for CD29, CD90, and CD54; over 85% of the cells are positive for CD73 and CD105; less than 6% of the cells are positive for CD14, CD19, CD31, CD34, CD39, CD45RA, HLA-DR, GlyA, CD200, and GlyA; and less than 20% of the cells are positive for SSEA4. The aforementioned, non-limiting marker expression patterns were found in certain maternal placental cell populations that were expanded on 3D substrates.

In other embodiments, each of CD73, CD29, and CD105 is expressed by more than 90% of the ASC; and over 90% (or in other embodiments, over 95%, or in other embodiments, over 98%) of the cells do not differentiate into adipocytes, under conditions where mesenchymal stem cells differentiate into adipocytes. In some embodiments, as provided herein, the conditions are incubation of adipogenesis induction medium, for example a solution containing 1 mcM dexamethasone, 0.5 mM 3-Isobutyl-1-methylxanthine (IBMX), 10 mcg/ml insulin, and 100 mcM indomethacin, on days 1, 3, 5, 9, 11, 13, 17, 19, and 21; and replacement of the medium with adipogenesis maintenance medium, namely a solution containing 10 mcg/ml insulin, on days 7 and 15, for a total of 25 days (“standard adipogenesis induction conditions”). In yet other embodiments, each of CD34, CD45, CD19, CD14 and HLA-DR is expressed by less than 3% of the cells; and the cells do not differentiate into adipocytes, after incubation under the aforementioned conditions. In other embodiments, each of CD73, CD29, and CD105 is expressed by more than 90% of the cells, each of CD34, CD45, CD19, CD14 and HLA-DR is expressed by less than 3% of the cells; and the cells do not differentiate into adipocytes, after incubation under the aforementioned conditions. In still other embodiments, a modified adipogenesis induction medium, containing 1 mcM dexamethasone, 0.5 mM IBMX, 10 mcg/ml insulin, and 200 mcM indomethacin is used, and the incubation is for a total of 26 days (“modified adipogenesis induction conditions”). The aforementioned solutions will typically contain cell culture medium such as DMEM+10% serum or the like, as will be appreciated by those skilled in the art. The aforementioned, non-limiting phenotypes and marker expression patterns were found in certain maternal placental cell populations that were expanded on 3D substrates.

In yet other embodiments, the placental MSC do not express Neutrophil gelatinase-associated lipocalin (LCN2; Uniprot Accession No. P80188).

“Positive” expression of a marker indicates a value higher than the range of the main peak of an isotype control histogram; this term is synonymous herein with characterizing a cell as “express”/“expressing” a marker. “Negative” expression of a marker indicates a value falling within the range of the main peak of an isotype control histogram; this term is synonymous herein with characterizing a cell as “not express”/“not expressing” a marker. “High” expression of a marker, and term “highly express[es]” indicates an expression level that is more than 2 standard deviations higher than the expression peak of an isotype control histogram, or a bell-shaped curve matched to said isotype control histogram. Reference herein to “secrete”/“secreting”/“secretion” relates to a detectable secretion of the indicated factor, above background levels in standard assays. For example, 0.5×10⁶ fetal or maternal ASC can be suspended in 4 ml medium (DMEM+10% fetal bovine serum (FBS)+2 mM L-Glutamine), added to each well of a 6 well-plate, and cultured for 24 hrs in a humidified incubator (5% C02, at 37° C.). After 24 h, DMEM is removed, and cells are cultured for an additional 24 hrs in 1 ml RPMI 1640 medium+2 mM L-Glutamine+0.5% HSA. The CM is collected from the plate, and cell debris is removed by centrifugation.

In still other embodiments, the majority, in other embodiments over 60%, over 70%, over 80%, or over 90% of the expanded cells express CD29, CD73, CD90, and CD105. In yet other embodiments, less than 20%, 15%, or 10% of the described cells express CD3, CD4, CD34, CD39, and CD106. In yet other embodiments, less than 20%, 15%, or 10% of the described cells highly express CD56. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, more than 50% of the cells express, or in other embodiments highly express, CD141 (thrombomodulin; UniProt Accession No. P07204), or in other embodiments SSEA4 (stage-specific embryonic antigen 4, an epitope of ganglioside GL-7 (IV³ NeuAc 2→3 GalGB4); Kannagi R et at), or in other embodiments both markers. Alternatively or in addition, more than 50% of the cells express HLA-A2 (UniProt Accession No. P01892). The aforementioned, non-limiting marker expression patterns were found in certain fetally-derived placental cell populations that were expanded on 3D substrates. The Uniprot Accession Nos. mentioned in the paragraph were accessed on accessed on Feb. 8, 2017.

In other embodiments, each of CD29, CD73, CD90, and CD105 is expressed by more than 80% of the cells that have been expanded; and over 90% (or in other embodiments, over 95%, or in other embodiments, over 98%) of the cells do not differentiate into osteocytes, after incubation for 17 days with a solution containing 0.1 mcM dexamethasone, 0.2 mM ascorbic acid, and 10 mM glycerol-2-phosphate, in plates coated with vitronectin and collagen (“standard osteogenesis induction conditions”). In yet other embodiments, each of CD34, CD39, and CD106 is expressed by less than 10% of the cells; less than 20% of the cells highly express CD56; and the cells do not differentiate into osteocytes, after incubation under the aforementioned conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56, and/or the cells do not differentiate into osteocytes, after incubation under the aforementioned conditions. In still other embodiments, the conditions are incubation for 26 days with a solution containing 10 mcM dexamethasone, 0.2 mM ascorbic acid, 10 mM glycerol-2-phosphate, and 10 nM Vitamin D, in plates coated with vitronectin and collagen (“modified osteogenesis induction conditions”). The aforementioned solutions will typically contain cell culture medium such as DMEM+10% serum or the like, as will be appreciated by those skilled in the art. In yet other embodiments, less than 20%, 15%, or 10% of the described cells highly express CD56. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetally-derived placental cell populations that were expanded on 3D substrates.

In other embodiments, each of CD29, CD73, CD90, and CD105 is expressed by more than 80% of the cells that have been expanded; and over 90% (or in other embodiments, over 95%, or in other embodiments, over 98%) of the cells do not differentiate into adipocytes, under standard adipogenesis induction conditions. In yet other embodiments, each of CD34, CD39, and CD106 is expressed by less than 10% of the cells; less than 20% of the cells highly express CD56; and the cells do not differentiate into adipocytes under the aforementioned conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56; and the cells do not differentiate into adipocytes, after incubation under the aforementioned conditions. In still other embodiments, modified adipogenesis induction conditions are used. In still other embodiments, over 90% of the cells in each population do not differentiate into either adipocytes or osteocytes under the aforementioned standard conditions. In yet other embodiments, over 90% of the cells in each population do not differentiate into either adipocytes or osteocytes under the aforementioned modified conditions. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetally-derived placental cell populations that were expanded on 3D substrates.

In still other embodiments, the placental ASC do not express TGFB3 (Transforming growth factor, beta 3; Uniprot Accession No. A5YM40) at a significant level. In other embodiments, the placental ASC do not express BMP2 (Bone morphogenetic protein 2; Uniprot Accession No. P12643) at a significant level. Expression at a significant level may refer, in some embodiments, to expression at least 50% above background levels. In other embodiments, the placental ASC, after culturing on a 3D substrate (e.g. culturing for 5 days on a polyester non-woven fibrous matrix), express TGFB3 at a level at least 2-fold, 3-fold, 4-fold, 5-fold, 6-fold, 8-fold, 10-fold, 12-fold, 15-fold, or 20-fold lower than BM-MSC cultured under the same conditions. In still other embodiments, the placental ASC, after culturing on a 3D substrate (e.g. culturing for 5 days on a polyester non-woven fibrous matrix), express BMP2 at a level at least 2-fold, 3-fold, 4-fold, 5-fold, 6-fold, 8-fold, 10-fold, 12-fold, 15-fold, or 20-fold lower than BM-MSC cultured under the same conditions. Uniprot Accession Numbers in this paragraph were accessed on Feb. 5, 2018. One or more of the aforementioned expression levels may be freely combined with any of the embodiments of expression of surface markers described herein.

Additionally or alternatively, the ASC secrete or express (as appropriate in each case) IL-6 (UniProt identifier P05231), IL-8 (UniProt identifier P10145), eukaryotic translation elongation factor 2 (EEEF2), reticulocalbin 3, EF-hand calcium binding domain (RCN2), and/or calponin 1 basic smooth muscle (CNN1). In more specific embodiments, greater than 50%, in other embodiments greater than 55%, in other embodiments greater than 60%, in other embodiments greater than 65%, in other embodiments greater than 70%, in other embodiments greater than 75%, in other embodiments greater than 80%, in other embodiments greater than 85%, in other embodiments greater than 90%, in other embodiments greater than 95%, in other embodiments greater than 96%, in other embodiments greater than 97%, in other embodiments greater than 98%, in other embodiments greater than 99%, of the cells express or secrete at least one, in other embodiments at least 2, in other embodiments at least 3, in other embodiments at least 4, in other embodiments all five of the aforementioned proteins.

In still other embodiments, the ASC secrete Flt-3 ligand (Fms-related tyrosine kinase 3 ligand; Uniprot Accession No. P49772), stem cell factor (SCF; Uniprot Accession No. P21583), IL-6 (Interleukin-6; UniProt identifier P05231), or combinations thereof, each of which represents a separate embodiment. In certain embodiments, the ASC secrete levels of Flt-3 ligand, SCF, IL-6, or in other embodiments combinations thereof, that are at least 2-, 3-, 4-, 5-, 6-, 8-, 10, 12-, 15-, or 20-fold higher than that expressed or secreted by ASC of placenta or adipose tissue grown on a 2D substrate. ASC grown on a 3D substrate secrete higher levels of Flt-3 ligand, SCF, and IL-6 than ASC grown on a 2D substrate, as provided in PCT Application Publ. No. WO/2007/108003, which is fully incorporated herein by reference in its entirety. Uniprot entries in this and the following 2 paragraphs were accessed on Feb. 26, 2017.

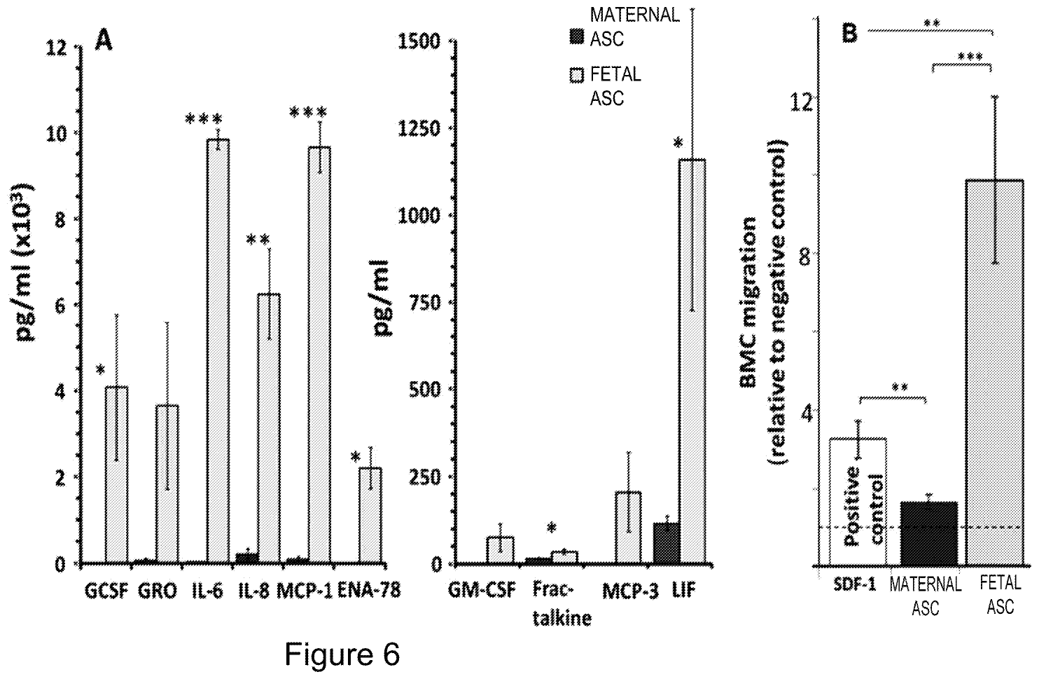

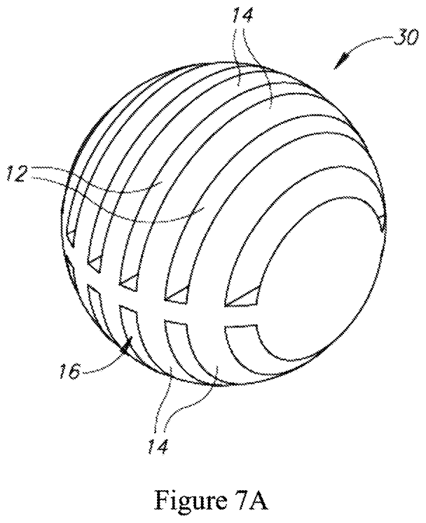

In yet other embodiments, the ASC secrete G-CSF (Granulocyte colony-stimulating factor; Uniprot Accession No. P09919); GM-CSF (Granulocyte-macrophage colony-stimulating factor; Uniprot Accession No. P04141); GRO (CXCL1; Uniprot Accession No. P09341); IL-6; IL-8; MCP-1, MCP-3 (Monocyte chemoattractant proteins 1 and 3/UniProt Nos. P13500 and P80098, respectively), ENA-78 (CXCL5; Uniprot Accession No. P42830); LIF (Leukemia inhibitory factor; Uniprot Accession No. P15018); or a combination thereof, each of which represents a separate embodiment. In certain embodiments, the ASC secrete levels of G-CSF, GM-CSF, GRO; IL-6; IL-8; MCP-1, MCP-3, ENA-78, or LIF, or in other embodiments any combination of some or all of these factors, that are at least 2-, 3-, 4-, 5-, 6-, 8-, 10, 12-, 15-, or 20-fold higher than maternally-derived cell populations. As provided herein, fetal ASC grown on a 3D substrate secrete higher levels of these factors than maternal ASC populations grown under similar conditions (FIG. 6).

In other embodiments, the ASC secrete EPO (Erythropoietin; UniProt identifier P01588), IL-3 (interleukin-3; Uniprot Accession No. P08700), IL-6, SCF, or combinations thereof, each of represents a separate embodiment. In certain embodiments, the ASC secrete levels of EPO, IL-3, IL-6, SCF, or in other embodiments of a combination thereof, that are at least 2-, 3-, 4-, 5-, 6-, 8-, 10, 12-, 15-, or 20-fold higher than maternally-derived cell populations. As provided herein, fetal ASC grown on a 3D substrate secrete higher levels of EPO, IL-3, IL-6, and SCF than maternal ASC populations grown under similar conditions.

In still other embodiments, the ASC increase levels of RBC, WBC, or platelets in the subject with hematopoietic dysfunction; or in other embodiments at least 2 thereof, each of which represents a separate embodiment; in or in other embodiments levels of RBC, WBC, and platelets. In yet other embodiments, the ASC induce secretion of KC ((keratinocyte chemoattractant/CXCL1; Uniprot No. P09341), IL-6, and GM-CSF in the serum and/or BM of irradiated subjects. The described ASC, in particular fetally-derived ASC, induce secretion of KC, IL-6, and GM-CSF in the serum and BM, and increase levels of RBC, WBC, and platelets, when administered to subjects with hematological deficiencies, as provided in PCT Publication No. WO/2016/151476 to Zami Aberman, which is incorporated herein by reference in its entirety.

According to some embodiments, the described ASC are capable of suppressing an immune reaction in the subject. Methods of determining the immunosuppressive capability of a cell population are well known to those skilled in the art, and exemplary methods are described in Example 3 of PCT Publication No. WO 2009/144720, which is incorporated herein by reference in its entirety. For example, a mixed lymphocyte reaction (MLR) may be performed. In an exemplary, non-limiting MLR assay, irradiated cord blood (iCB) cells, for example human cells or cells from another species, are incubated with peripheral blood-derived monocytes (PBMC; for example human PBMC or PBMC from another species), in the presence or absence of a cell population to be tested. PBMC cell replication, which correlates with the intensity of the immune response, can be measured by a variety of methods known in the art, for example by ³H-thymidine uptake. Reduction of the PBMC cell replication when co-incubated with test cells indicates an immunosuppressive capability. Alternatively, a similar assay can be performed with peripheral blood (PB)-derived MNC, in place of CB cells. Alternatively or in addition, secretion of pro-inflammatory and anti-inflammatory cytokines by blood cell populations (such as CB cells or PBMC) can be measured when stimulated (for example by incubation with non-matched cells, or with a non-specific stimulant such as PHA), in the presence or absence of the ASC. In certain embodiments, for example in the case of human ASC, as provided in WO 2009/144720, when 150,000 ASC are co-incubated for 48 hours with 50,000 allogeneic PBMC, followed by a 5-hour stimulation with 1.5 mcg/ml of LPS, the amount of IL-10 secretion by the PBMC is at least 120%, at least 130%, at least 150%, at least 170%, at least 200%, or at least 300% of the amount observed with LPS stimulation in the absence of ASC.

In other embodiments, each of CD73, CD29, and CD105 is expressed by more than 90% of the described ASC; and over 90% (or in other embodiments, over 95%, or in other embodiments, over 98%) of the cells inhibit T cell proliferation. In yet other embodiments, each of CD34, CD19, and CD14 is expressed by less than 3% of the cells; and the cells inhibit T cell proliferation. In other embodiments, each of CD73, CD29, and CD105 is expressed by more than 90% of the cells, each of CD34, CD19, and CD14 is expressed by less than 3% of the cells; and the cells inhibit T cell proliferation.

In still other embodiments, the ASC secrete immunoregulatory factor(s). In certain embodiments, the ASC secrete a factor selected from TNF-beta (UniProt identifier P01374) and Leukemia inhibitory factor (LIF; UniProt identifier P15018). In other embodiments, the ASC secrete a factor selected from MCP-1 (CCL2), Osteoprotegerin, MIF (Macrophage migration inhibitory factor; Uniprot Accession No. P14174), GDF-15, SDF-1 alpha, GROa (Growth-regulated alpha protein; Uniprot Accession No. P09341), beta2-Microglobulin, IL-6, IL-8 (UniProt identifier P10145), TNF-beta, ENA78/CXCL5, eotaxin/CCL11 (Uniprot Accession No. P51671), and MCP-3 (CCL7). In certain embodiments, the ASC secrete MCP-1, Osteoprotegerin, MIF, GDF-15, SDF-1 alpha, GROa, beta2-Microglobulin, IL-6, IL-8, TNF-beta, and MCP-3, which were found to be secreted by maternal cells. In other embodiments, the ASC secrete MCP-1, Osteoprotegerin, MIF, GDF-15, SDF-1 alpha, beta2-Microglobulin, IL-6, IL-8, ENA78, eotaxin, and MCP-3, which were found to be secreted by fetal cells. All Swissprot and UniProt entries in this paragraph were accessed on Mar. 23, 2017.

In other embodiments, the described ASC exhibit a spindle shape when cultured under 2D conditions.

According to some embodiments, the ASC express CD200, while in other embodiments, the ASC lack expression of CD200. In still other embodiments, less than 30%, 25%, 20%, 15%, 10%, 8%, 6%, 5%, 4%, 3%, or 2%, 1%, or 0.5% of the adherent cells express CD200. In yet other embodiments, greater than 70%, 75%, 80%, 85%, 90%, 92%, 94%, 95%, 96%, 97%, 98%, 99%, or 99.5% of the adherent cells express CD200.

In still other embodiments, the cells may be allogeneic, or in other embodiments, the cells may be autologous. In other embodiments, the cells may be fresh or, in other embodiments, frozen (for example, cryo-preserved).

Additional Method Characteristics for Preparation of ASC

In certain embodiments, the described ASC have been subject to a 3D incubation, as described further herein. In more specific embodiments, the ASC have been incubated in a 2D adherent-cell culture apparatus, prior to the step of 3D culturing. In some embodiments, cells (which have been extracted, in some embodiments, from placenta, from adipose tissue, etc.) are then subjected to prior step of incubation in a 2D adherent-cell culture apparatus, followed by the described 3D culturing steps.

The terms “two-dimensional culture” and “2D culture” refer to a culture in which the cells are exposed to conditions that are compatible with cell growth and allow the cells to grow in a monolayer, which is referred to as a “2D culture apparatus”. Such apparatuses will typically have flat growth surfaces (also referred to as a “two-dimensional substrate(s)” or “2D substrate(s)”), in some embodiments comprising an adherent material, which may be flat or curved. Non-limiting examples of apparatuses for 2D culture are cell culture dishes and plates. Included in this definition are multi-layer trays, such as Cell Factory™, manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

The terms “three-dimensional culture” and “3D culture” refer to a culture in which the cells are exposed to conditions that are compatible with cell growth and allow the cells to grow in a 3D orientation relative to one another. The term “three-dimensional [or 3D] culture apparatus” refers to an apparatus for culturing cells under conditions that are compatible with cell growth and allow the cells to grow in a 3D orientation relative to one another. Such apparatuses will typically have a 3D growth surface (also referred to as a “three-dimensional substrate” or “3D substrate”), in some embodiments comprising an adherent material, which is present in the 3D culture apparatus, e.g. the bioreactor. Certain, non-limiting embodiments of 3D culturing conditions suitable for expansion of adherent stromal cells are described in PCT Application Publ. No. WO/2007/108003, which is fully incorporated herein by reference in its entirety.

In various embodiments, “an adherent material” refers to a material that is synthetic, or in other embodiments naturally occurring, or in other embodiments a combination thereof. In certain embodiments, the material is non-cytotoxic (or, in other embodiments, is biologically compatible). Alternatively or in addition, the material is fibrous, which may be, in more specific embodiments, a woven fibrous matrix, a non-woven fibrous matrix, or any type of fibrous matrix. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. Other embodiments include Matrigel™, an extra-cellular matrix component (e.g., Fibronectin, Chondronectin, Laminin), and a collagen. In more particular embodiments, the material may be selected from a polyester and a polypropylene. Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids.

In other embodiments, the length of 3D culturing is at least 4 days; between 4-12 days; in other embodiments between 4-11 days; in other embodiments between 4-10 days; in other embodiments between 4-9 days; in other embodiments between 5-9 days; in other embodiments between 5-8 days; in other embodiments between 6-8 days; or in other embodiments between 5-7 days. In other embodiments, the 3D culturing is performed for 5-15 cell doublings, in other embodiments 5-14 doublings, in other embodiments 5-13 doublings, in other embodiments 5-12 doublings, in other embodiments 5-11 doublings, in other embodiments 5-10 doublings, in other embodiments 6-15 cell doublings, in other embodiments 6-14 doublings, in other embodiments 6-13 doublings, or in other embodiments 6-12 doublings, in other embodiments 6-11 doublings, or in other embodiments 6-10 doublings.

In certain embodiments, 3D culturing can be performed in a 3D bioreactor. In some embodiments, the 3D bioreactor comprises a container for holding medium and a 3D attachment substrate disposed therein, and a control apparatus, for controlling pH, temperature, and oxygen levels and optionally other parameters. The terms attachment substrate and growth substrate are interchangeable. In certain embodiments, the attachment substrate is in the form of carriers, which comprise, in more specific embodiments, a surface comprising a synthetic adherent material. Alternatively or in addition, the bioreactor contains ports for the inflow and outflow of fresh medium and gases. Except where indicated otherwise, the term “bioreactor” excludes decellularized organs and tissues derived from a living being.

Examples of bioreactors include, but are not limited to, a continuous stirred tank bioreactor, a CelliGen Plus® bioreactor system (New Brunswick Scientific (NBS) and a BIOFLO 310 bioreactor system (New Brunswick Scientific (NBS).

As provided herein, a 3D bioreactor is capable, in certain embodiments, of 3D expansion of ASC under controlled conditions (e.g. pH, temperature and oxygen levels) and with growth medium perfusion, which in some embodiments is constant perfusion and in other embodiments is adjusted in order to maintain target levels of glucose or other components. Furthermore, the cell cultures can be directly monitored for concentrations of glucose, lactate, glutamine, glutamate and ammonium. The glucose consumption rate and the lactate formation rate of the adherent cells enable, in some embodiments, measurement of cell growth rate and determination of the harvest time.

In some embodiments, a continuous stirred tank bioreactor is used, where a culture medium is continuously fed into the bioreactor and a product is continuously drawn out, to maintain a time-constant steady state within the reactor. A stirred tank bioreactor with a fibrous bed basket is available for example from New Brunswick Scientific Co., Edison, N.J.). Additional bioreactors that may be used, in some embodiments, are stationary-bed bioreactors; and air-lift bioreactors, where air is typically fed into the bottom of a central draught tube flowing up while forming bubbles, and disengaging exhaust gas at the top of the column. Additional possibilities are cell-seeding perfusion bioreactors with polyactive foams [as described in Wendt, D. et al., Biotechnol Bioeng 84: 205-214, (2003)] and radial-flow perfusion bioreactors containing tubular poly-L-lactic acid (PLLA) porous scaffolds [as described in Kitagawa et al., Biotechnology and Bioengineering 93(5): 947-954 (2006). Other bioreactors which can be used are described in U.S. Pat. Nos. 6,277,151; 6,197,575; 6,139,578; 6,132,463; 5,902,741; and 5,629,186, which are incorporated herein by reference. A “stationary-bed bioreactor” refers to a bioreactor in which the cellular growth substrate is not ordinarily lifted from the bottom of the incubation vessel in the presence of growth medium. For example, the substrate may have sufficient density to prevent being lifted and/or it may be packed by mechanical pressure to present it from being lifted. The substrate may be either a single body or multiple bodies. Typically, the substrate remains substantially in place during perfusion at the bioreactor's standard perfusion rate. In certain embodiments, the substrate may be lifted at unusually fast perfusion rates, for example greater than 200 rpm.

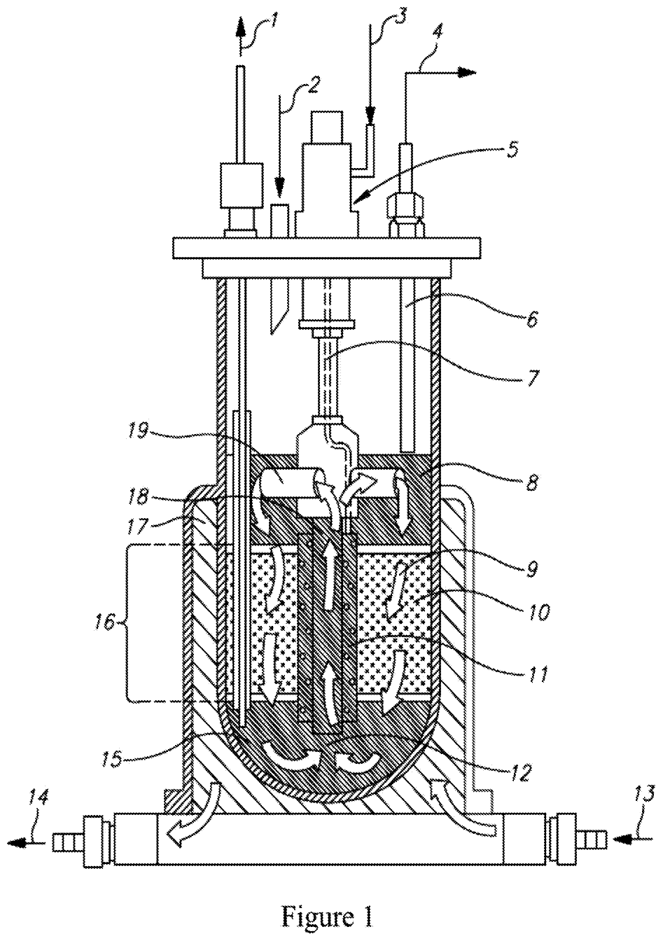

Another exemplary, non-limiting bioreactor, the Celligen 310 Bioreactor, is depicted in FIG. 1. A Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow stirring initial rate is used to promote cell attachment, then agitation is increased. Alternatively or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively or in addition, sparged gas confined to the remote chamber is absorbed by the nutrient medium, which washes over the immobilized cells. In still other embodiments, a water jacket (17) is present, with ports for moving the jacket water in (13) and out (14).

In certain embodiments, a perfused bioreactor is used, wherein the perfusion chamber contains carriers. The carriers may be, in more specific embodiments, selected from macrocarriers, microcarriers, or either. Non-limiting examples of microcarriers that are available commercially include alginate-based (GEM, Global Cell Solutions), dextran-based (Cytodex, GE Healthcare), collagen-based (Cultispher, Percell), and polystyrene-based (SoloHill Engineering) microcarriers. In certain embodiments, the microcarriers are packed inside the perfused bioreactor.

In some embodiments, the carriers in the perfused bioreactor are packed, for example forming a packed bed, which is submerged in a nutrient medium. Alternatively or in addition, the carriers may comprise an adherent material. In other embodiments, the surface of the carriers comprises an adherent material, or the surface of the carriers is adherent. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. In more particular embodiments, the material may be selected from a polyester and a polypropylene. In various embodiments, an “adherent material” refers to a material that is synthetic, or in other embodiments naturally occurring, or in other embodiments a combination thereof. In certain embodiments, the material is non-cytotoxic (or, in other embodiments, is biologically compatible). Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids. Other embodiments include Matrigel™, an extra-cellular matrix component (e.g., Fibronectin, Chondronectin, Laminin), and a collagen.

In other embodiments, cells are produced using a packed-bed spinner flask. In more specific embodiments, the packed bed may comprise a spinner flask and a magnetic stirrer. The spinner flask may be fitted, in some embodiments, with a packed bed apparatus, which may be, in more specific embodiments, a fibrous matrix; or in more specific embodiments, a non-woven fibrous matrix. In other embodiments, the fibrous matrix comprises polyester, or comprises at least about 50% polyester. In still other embodiments, the non-woven fibrous matrix comprises polyester, or comprises at least about 50% polyester.

In still other embodiments, the matrix is similar to the Celligen™ Plug Flow bioreactor which is, in certain embodiments, packed with Fibra-cel® carriers (or, in other embodiments, other carriers). The spinner is, in certain embodiments, batch fed (or in other alternative embodiments fed by perfusion), fitted with one or more sterilizing filters, and placed in a tissue culture incubator. In further embodiments, cells are seeded onto the scaffold by suspending them in medium and introducing the medium to the apparatus. In still further embodiments, the agitation speed is gradually increased, for example by starting at 40 RPM for 4 hours, then gradually increasing the speed to 120 RPM. In certain embodiments, the glucose level of the medium may be tested periodically (i.e. daily), and the perfusion speed adjusted maintain an acceptable glucose concentration, which is, in certain embodiments, between 400-700 mg\liter, between 450-650 mg\liter, between 475-625 mg\liter, between 500-600 mg\liter, or between 525-575 mg\liter. In yet other embodiments, at the end of the culture process, carriers are removed from the packed bed, washed with isotonic buffer, and processed or removed from the carriers by agitation and/or enzymatic digestion.

In certain embodiments, the bioreactor is seeded at a concentration of between 10,000-2,000,000 cells/ml (milliliter) of medium; or, in other embodiments, within one of the following ranges: 20,000-2,000,000, 30,000-1,500,000, 40,000-1,400,000, 50,000-1,300,000, 60,000-1,200,000, 70,000-1,100,000, 80,000-1,000,000, 80,000-900,000, 80,000-800,000, 80,000-700,000, 80,000-600,000, 80,000-500,000, 80,000-400,000, 90,000-300,000, 90,000-250,000, 90,000-200,000, 100,000-200,000, 110,000-1,900,000, 120,000-1,800,000, 130,000-1,700,000, or 140,000-1,600,000 cells/ml of medium.

In still other embodiments, between 1-20×10⁶ cells per gram (gr) of carrier (substrate) are seeded or, in other embodiments, within one of the following ranges: 1.5-20×10⁶ cells/gr carrier, 1.5-18×10⁶, 1.8-18×10⁶, 2-18×10⁶, 3-18×10⁶, 2.5-15×10⁶, 3-15×10⁶, 3-14×10⁶, 3-12×10⁶, 3.5-12×10⁶, 3-10×10⁶, 3-9×10⁶, 4-9×10⁶, 4-8×10⁶, 4-7×10⁶, or 4.5-6.5×10⁶ cells/gr carrier.

In certain embodiments, the harvest from the bioreactor is performed when at least about 10%, in other embodiments at least 12%, in other embodiments at least 14%, in other embodiments at least 16%, in other embodiments at least 18%, in other embodiments at least 20%, in other embodiments at least 22%, in other embodiments at least 24%, in other embodiments at least 26%, in other embodiments at least 28%, or in other embodiments at least 30% of the cells are in the S and G2/M phases (collectively), as can be assayed by various methods known in the art, for example FACS detection. Typically, in the case of FACS, the percentage of cells in S and G2/M phase is expressed as the percentage of the live cells, after gating for live cells, for example using a forward scatter/side scatter gate. Those skilled in the art will appreciate that the percentage of cells in these phases correlates with the percentage of proliferating cells. In some cases, allowing the cells to remain in the bioreactor significantly past their logarithmic growth phase causes a reduction in the number of cells that are proliferating.

In other embodiments, over 5×10⁵, over 7×10⁵, over 8×10⁵, over 9×10⁵, over 10⁶, over 1.5×10⁶, over 2×10⁶, over 3×10⁶, over 4×10⁶, or over 5×10⁶ viable cells are removed per milliliter of the growth medium in the bioreactor. In still other embodiments over between 5×10⁵-1.5×10⁷, between 7×10⁵-1.5×10⁷, between 8×10⁵-1.5×10⁷, between 1×10⁶-1.5×10⁷, between 5×10⁵-1×10⁷, between 7×10⁵-1×10⁷, between 8×10⁵-1×10⁷, between 1×10⁶-1×10⁷, between 1.2×10⁶-1×10⁷, or between 2×10⁶-1×10⁷ viable cells are removed per milliliter of the growth medium in the bioreactor.

In other embodiments, incubation of ASC may comprise microcarriers, which may, in certain embodiments, be inside a bioreactor. Microcarriers are known to those skilled in the art, and are described, for example in U.S. Pat. Nos. 8,828,720, 7,531,334, 5,006,467, which are incorporated herein by reference. Microcarriers are also commercially available, for example as Cytodex™ (available from Pharmacia Fine Chemicals, Inc.), Superbeads (commercially available from Flow Labs, Inc.), and DE-52 and DE-53 (commercially available from Whatman, Inc.). In certain embodiments, the ASC may be incubated in a 2D apparatus, for example tissue culture plates or dishes, prior to incubation in microcarriers. In other embodiments, the ASC are not incubated in a 2D apparatus prior to incubation in microcarriers. In certain embodiments, the microcarriers are packed inside a bioreactor.

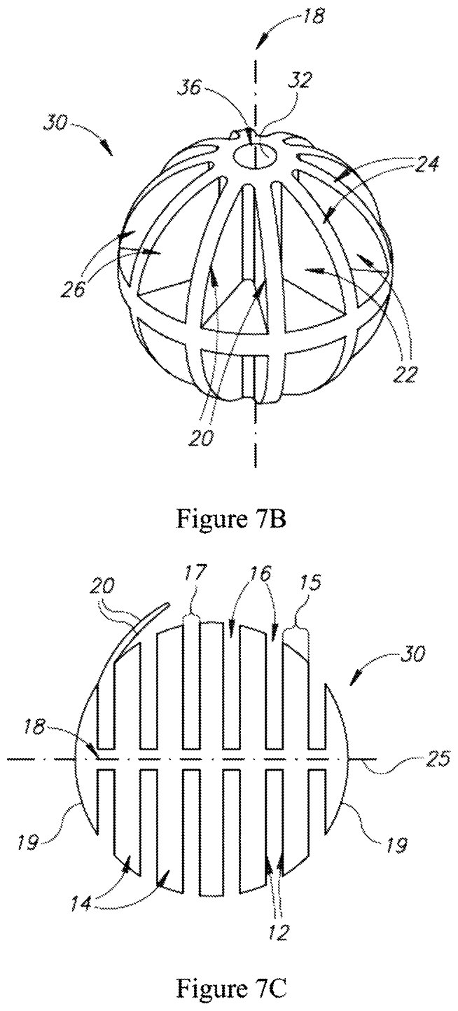

In some embodiments, with reference to FIGS. 7A-B, and as described in WO/2014/037862, published on Mar. 13, 2014, which is incorporated herein by reference in its entirety, grooved carriers 30 are used for proliferation and/or incubation of ASC. In various embodiments, the carriers may be used following a 2D incubation (e.g. on culture plates or dishes), or without a prior 2D incubation. In other embodiments, incubation on the carriers may be followed by incubation on a 3D substrate in a bioreactor, which may be, for example, a packed-bed substrate or microcarriers; or incubation on the carriers may not be followed by incubation on a 3D substrate. Carriers 30 can include multiple two-dimensional (2D) surfaces 12 extending from an exterior of carrier 30 towards an interior of carrier 30. As shown, the surfaces are formed by a group of ribs 14 that are spaced apart to form openings 16, which may be sized to allow flow of cells and culture medium (not shown) during use. With reference to FIG. 7C, carrier 30 can also include multiple 2D surfaces 12 extending from a central carrier axis 18 of carrier 30 and extending generally perpendicular to ribs 14 that are spaced apart to form openings 16, creating multiple 2D surfaces 12. In some embodiments, carriers 30 are “3D bodies” as described in WO/2014/037862; the contents of which relating to 3D bodies are incorporated herein by reference.

In certain embodiments, the described carriers (e.g. grooved carriers) are used in a bioreactor. In some, the carriers are in a packed conformation.

In still other embodiments, the material forming the multiple 2D surfaces comprises at least one polymer. Suitable coatings may, in some embodiments, be selected to control cell attachment or parameters of cell biology.

In certain embodiments, further steps of purification or enrichment for ASC may be performed. Such methods include, but are not limited to, cell sorting using markers for ASC and/or, in various embodiments, mesenchymal stromal cells or mesenchymal-like ASC.

Cell sorting, in this context, refers to any procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

In more particular embodiments, cells may be removed from a 3D matrix while the matrix remains within the bioreactor. In certain embodiments, at least about 10%, at least 12%, at least 14%, at least 16%, at least 18%, at least 20%, at least 22%, at least 24%, at least 26%, at least 28%, or at least 30% of the cells are in the S and G2/M phases (collectively), at the time of harvest from the bioreactor. Cell cycle phases can be assayed by various methods known in the art, for example FACS detection. Typically, in the case of FACS, the percentage of cells in S and G2/M phase is expressed as the percentage of the live cells, after gating for live cells, for example using a forward scatter/side scatter gate. Those skilled in the art will appreciate that the percentage of cells in these phases correlates with the percentage of proliferating cells. In some cases, allowing the cells to remain in the bioreactor significantly past their logarithmic growth phase causes a reduction in the number of cells that are proliferating.

In certain embodiments, the harvesting process comprises agitation. In certain embodiments, the agitation is vibration, for example as described in PCT International Application Publ. No. WO 2012/140519, which is incorporated herein by reference. In certain embodiments, to effect harvesting, the cells are agitated at 0.7-6 Hertz, or in other embodiments 1-3 Hertz, during, or in other embodiments during and after, treatment with a protease, optionally also comprising a calcium chelator. In certain embodiments, the carriers containing the cells are agitated at 0.7-6 Hertz, or in other embodiments 1-3 Hertz, while submerged in a solution or medium comprising a protease, optionally also comprising a calcium chelator. Non-limiting examples of a protease plus a calcium chelator are trypsin, or another enzyme with similar activity, optionally in combination with another enzyme, non-limiting examples of which are Collagenase Types I, II, III, and IV, with EDTA. Enzymes with similar activity to trypsin are well known in the art; non-limiting examples are TrypLE™, a fungal trypsin-like protease, and Collagenase, Types I, II, III, and IV, which are available commercially from Life Technologies. Enzymes with similar activity to collagenase are well known in the art; non-limiting examples are Dispase I and Dispase II, which are available commercially from Sigma-Aldrich. In still other embodiments, the cells are harvested by a process comprising an optional wash step, followed by incubation with collagenase, followed by incubation with trypsin. In various embodiments, at least one, at least two, or all three of the aforementioned steps comprise agitation. In more specific embodiments, the total duration of agitation during and/or after treatment with protease plus a calcium chelator is between 2-10 minutes, in other embodiments between 3-9 minutes, in other embodiments between 3-8 minutes, and in still other embodiments between 3-7 minutes. In still other embodiments, the cells are subjected to agitation at 0.7-6 Hertz, or in other embodiments 1-3 Hertz, during the wash step before the protease and calcium chelator are added. Alternatively or in addition, the ASC are expanded using an adherent material in a container, which is in turn disposed within a bioreactor chamber, and an apparatus is used to impart a reciprocating motion to the container relative to the bioreactor chamber, wherein the apparatus is configured to move the container in a manner causing cells attached to the adherent material to detach from the adherent material. In more specific embodiments, the vibrator comprises one or more controls for adjusting amplitude and frequency of the reciprocating motion. Alternatively or in addition, the adherent material is a 3D substrate, which comprises, in some embodiments, carriers comprising a synthetic adherent material.

Those skilled in the art will appreciate that a variety of isotonic buffers may be used for washing cells and similar uses. Hank's Balanced Salt Solution (HBSS; Life Technologies) is only one of many buffers that may be used.

Non-limiting examples of base media useful in 2D and 3D culturing include Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM), Yamane, IMEM-20, Glasgow Modification Eagle Medium (GMEM), Leibovitz L-15 Medium, McCoy's 5A Medium, Medium M199 (M199E—with Earle's sale base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. In certain embodiments, DMEM is used. These and other useful media are available from GIBCO, Grand Island, N.Y., USA and Biological Industries, Bet HaEmek, Israel, among others.

In some embodiments, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be fetal bovine serum, or in other embodiments another animal serum. In still other embodiments, the medium is serum-free.

Alternatively or in addition, the medium may be supplemented by growth factors, vitamins (e.g. ascorbic acid), cytokines, salts (e.g. B-glycerophosphate), steroids (e.g. dexamethasone) and hormones e.g., growth hormone, erythropoietin, thrombopoietin, interleukin 3, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

The various media described herein, i.e. the 2D growth medium and the 3D growth medium, may be independently selected from each of the described embodiments relating to medium composition. In various embodiments, any medium suitable for growth of cells in a standard tissue apparatus and/or a bioreactor may be used.

It will also be appreciated that in certain embodiments, when the described ASC are intended for administration to a human subject, the cells and the culture medium (e.g., with the above-described medium additives) are substantially xeno-free, i.e., devoid of any animal contaminants e.g., mycoplasma. For example, the culture medium can be supplemented with a serum-replacement, human serum and/or synthetic or recombinantly produced factors.

In other embodiments, conditioned medium (CM) derived from the described ASC is utilized in the described methods, for example post-incubation medium from the described tissue culture incubation or bioreactor incubation. In yet other embodiments, there is provided a pharmaceutical composition comprising the CM, which may be, in some embodiments, indicated for the described therapeutic indications. Those skilled in the art will appreciate that, in certain embodiments, various bioreactors may be used to prepare CM, including but not limited to plug-flow bioreactors, and stationary-bed bioreactors (Kompier R et al. Use of a stationary bed reactor and serum-free medium for the production of recombinant proteins in insect cells. Enzyme Microb Technol. 1991. 13(10):822-7.) For example, CM is produced as a by-product of the described methods for cell expansion. The CM in the bioreactor can be removed from the bioreactor or otherwise isolated. In other embodiments, the described expanded cells are removed from the bioreactor and incubated in another apparatus (a non-limiting example of which is a tissue culture apparatus), and CM from the cells is collected.

In yet other embodiments, extracellular vesicles, e.g. exosomes, secreted by the described ASC are used in the described methods and compositions. Methods of isolating exosomes are known in the art, and include, for example, immuno-magnetic isolation, for example as described in Clayton A et al, 2001; Mathias R A et al, 2009; and Crescitelli R et al, 2013.

In certain embodiments, the described methods comprise isolation of exosomes, for example as described in Conde-Vancells et al. and Koga et al., or the references cited therein. One such protocol, provided solely for purposes of exemplification, involved centrifuging samples for 30 min at 1500×g to remove large cellular debris. The resultant supernatants are subjected to filtration on 0.22 μm pore filters, followed by ultra-centrifugation at 10 000×g and 100 000×g for 30 and 60 min, respectively. The resulting pellets are suspended in PBS, pooled, and again ultracentrifuged at 100 000×g for 60 min. The final pellet (containing vesicles) is suspended in 150 μL of PBS, aliquoted and stored at −80° C. For higher-purity preparations, exosomes can be further purified on sucrose-containing gradients (e.g. a 30% sucrose cushion), e.g. as described in Théry C et al. Vesicle preparations are diluted in PBS and under-layered on top of a density cushion composed of pH-buffered 30% sucrose (optionally containing deuterium oxide (D20)), around pH 7.4, forming a visible interphase. The samples are ultracentrifuged at 100 000×g at 4° C. for 75 min in a swinging bucket rotor, and the gradient is withdrawn in aliquots from the bottom. Vesicles contained in the 30% sucrose/D20 cushion are collected, diluted in buffered solution, and optionally centrifuged at 100 000×g to concentrate the contents. Kits for exosome isolation are available commercially, non-limiting examples of which are ExoQuick® reagents, ExoMAX Opti enhancer, and ExoFLOW products, all of which can be obtained from System Biosciences (Palo Alto, Calif.).

In some embodiments, the exosomes or other extracellular vesicles are harvested from a 3D bioreactor in which the ASC have been incubated. Alternatively or in addition, the cells are cryopreserved, and then are thawed, after which the exosomes are isolated. In some embodiments, after thawing, the cells are cultured in 2D culture, from which the exosomes are harvested.

Pharmaceutical Compositions

The described ASC, or CM derived thereform, can be administered as a part of a pharmaceutical composition, e.g., that further comprises one or more pharmaceutically acceptable carriers. Hereinafter, the term “pharmaceutically acceptable carrier” refers to a carrier or a diluent. In some embodiments, a pharmaceutically acceptable carrier does not cause significant irritation to a subject. In some embodiments, a pharmaceutically acceptable carrier does not abrogate the biological activity and properties of administered cells. Examples, without limitations, of carriers are propylene glycol, saline, emulsions and mixtures of organic solvents with water. In some embodiments, the pharmaceutical carrier is an aqueous solution of saline.

In other embodiments, compositions are provided herein that comprise ASC or CM in combination with an excipient, e.g., a pharmacologically acceptable excipient. In further embodiments, the excipient is an osmoprotectant or cryoprotectant, an agent that protects cells from the damaging effect of freezing and ice formation, which may in some embodiments be a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and a carrier protein are present; in certain embodiments, the osmoprotectant and carrier protein may be the same compound. Alternatively or in addition, the composition is frozen. In more specific embodiments, DMSO may be present at a concentration of 2-5%; or, in other embodiments, 5-10%; or, in other embodiments, 2-10%, 3-5%, 4-6%; 5-7%, 6-8%, 7-9%, 8-10%. DMSO, in other embodiments, is present with a carrier protein, a non-limiting example of which is albumin, e.g. human serum albumin. The cells may be any embodiment of ASC mentioned herein, each of which is considered a separate embodiment.

Provided in addition are pharmaceutical compositions, comprising the described placental ASC, in the absence of non-placental cell types.

Also provided are pharmaceutical compositions, comprising the described placental ASC-derived CM, in the absence of CM derived from other cell types.

In other embodiments, there are provided pharmaceutical compositions, comprising the described exosomes.

Since non-autologous cells may in some cases induce an immune reaction when administered to a subject, several approaches may be utilized according to the methods provided herein to reduce the likelihood of rejection of non-autologous cells. In some embodiments, these approaches include either suppressing the recipient immune system or encapsulating the non-autologous cells in immune-isolating, semipermeable membranes before transplantation. In some embodiments, this may be done regardless of whether the ASC themselves engraft in the host. For example, the majority of the cells may, in various embodiments, not survive after engraftment for more than 3 days, more than 4 days, more than 5 days, more than 6 days, more than 7 days, more than 8 days, more than 9 days, more than 10 days, or more than 14 days.

In other embodiments, an immunosuppressive agent is present in the pharmaceutical composition. Examples of immunosuppressive agents that may be used in the methods and compositions provided herein include, but are not limited to, methotrexate, cyclophosphamide, cyclosporine, cyclosporine A, chloroquine, hydroxychloroquine, sulfasalazine (sulphasalazopyrine), gold salts, D-penicillamine, leflunomide, azathioprine, anakinra, infliximab (REMICADE), etanercept, TNF-alpha blockers, biological agents that antagonize one or more inflammatory cytokines, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, and tramadol.

One may, in various embodiments, administer the pharmaceutical composition in a systemic manner (as detailed hereinabove). Alternatively, one may administer the pharmaceutical composition locally, for example, via injection of the pharmaceutical composition directly into an exposed or affected tissue region of a patient. In other embodiments, the cells are administered intravenously (IV), subcutaneously (SC), by the intraosseous route (e.g. by intraosseous infusion), or intraperitoneally (IP), each of which is considered a separate embodiment. In other embodiments, the ASC or composition is administered intramuscularly; while in other embodiments, the ASC or composition is administered systemically. In this regard, “intramuscular” administration refers to administration into the muscle tissue of a subject; “subcutaneous” administration refers to administration just below the skin; “intravenous” administration refers to administration into a vein of a subject; “intraosseous” administration refers to administration directly into bone marrow; and “intraperitoneal” administration refers to administration into the peritoneum of a subject. In still other embodiments, the cells are administered intratracheally, intrathecally, by inhalational, or intranasally. In certain embodiments, lung-targeting routes of administration may utilize cells encapsulated in liposomes or other barriers to reduce entrapment within the lungs.

In still other embodiments, the pharmaceutical composition is administered intralymphatically, for example as described in U.S. Pat. No. 8,679,834 in the name of Eleuterio Lombardo and Dirk Buscher, which is hereby incorporated by reference.

In other embodiments, for injection, the described cells may be formulated in aqueous solutions, e.g. in physiologically compatible buffers such as Hank's solution, Ringer's solution, or physiological salt buffer, optionally in combination with medium containing cryopreservation agents.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

The data obtained from these in vitro and cell culture assays and animal studies can be used in formulating a range of dosage for use in human. The dosage may vary depending upon the dosage form employed and the route of administration utilized. The exact formulation, route of administration and dosage can be, in some embodiments, chosen by the individual physician in view of the patient's condition.

A typical dosage of the described ASC used alone ranges, in some embodiments, from about 10 million to about 500 million cells per administration. For example, the dosage can be, in some embodiments, 10, 20, 30, 40, 50, 60, 70, 80, 90, 100, 125, 150, 175, 200, 225, 250, 275, 300, 325, 350, 375, 400, 425, 450, 475, or 500 million cells or any amount in between these numbers. It is further understood that a range of ASC can be used including from about 10 to about 500 million cells, from about 100 to about 400 million cells, from about 150-300 million cells. Accordingly, disclosed herein are therapeutic methods, the method comprising administering to a subject a therapeutically or prophylactically effective amount of ASC, wherein the dosage administered to the subject is 10, 20, 30, 40, 50, 60, 70, 80, 90, 100, 125, 150, 175, 200, 225, 250, 275, 300, 325, 350, 375, 400, 425, 450, 475, or 500 million cells or, in other embodiments, between 150 million to 300 million cells. ASC, compositions comprising ASC, and/or medicaments manufactured using ASC can be administered, in various embodiments, in a series of 1, 2, 3, 4, 5, 6,7, 8,9, 10, 11, 12, 13, 14, 15, 1-10, 1-15, 1-20, 2-10, 2-15, 2-20, 3-20, 4-20, 5-20, 5-25, 5-30, 5-40, or 5-50 injections, or more.

Depending on the severity and responsiveness of the condition to be treated, dosing can be of a single or, in other embodiments, a plurality of administrations, with a course of treatment lasting from several days to several weeks or, in other embodiments, until alleviation of the disease state is achieved.

In certain embodiments, following administration, the majority of the cells, in other embodiments more than 60%, more than 70%, more than 80%, more than 90%, more than 95%, more than 96%, more than 97%, more than 98%, or more than 99% of the cells are no longer detectable within the subject 1 month after administration.

Compositions including the described preparations formulated in a compatible pharmaceutical carrier may also be prepared, placed in an appropriate container, and labeled for treatment of an indicated condition.

The described compositions may, if desired, be packaged in a container that is accompanied by instructions for administration. The container may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

In other embodiments, the described ASC are suitably formulated as a pharmaceutical composition which can be suitably packaged as an article of manufacture. Such an article of manufacture comprises a packaging material which comprises a label describing a use in treating a disease or disorder or therapeutic indication that is mentioned herein. In other embodiments, a pharmaceutical agent is contained within the packaging material, wherein the pharmaceutical agent is effective for the treatment of a disorder or therapeutic indication that is mentioned herein. In some embodiments, the pharmaceutical composition is frozen.

It is clarified that each embodiment of the described ASC may be freely combined with each embodiment relating to a therapeutic method or pharmaceutical composition. Furthermore, the cells utilized in the method or contained in the composition can be, in various embodiments, autologous, allogeneic, or xenogenic to the treated subject. Each type of cell may be freely combined with the therapeutic embodiments mentioned herein.

Furthermore, each embodiment of the described exosomes may be freely combined with each embodiment relating to a therapeutic method or pharmaceutical composition.

In still other embodiments, the described CM is used in any of the described therapeutic methods. Each embodiment of CM may be freely combined with each embodiment relating to a therapeutic method or pharmaceutical composition.

Subjects

In certain embodiments, the subject treated by the described methods and compositions has been exposed to a toxic chemical agent, which may be, in some embodiments, a vesicant, or in other embodiments, an organophosphate, or in other embodiments, a nerve agent. In certain embodiments, the chemical agent is a gas. In other embodiments, the chemical agent is a liquid. In certain embodiments, the subject is a human. In other embodiments, the subject may be an animal. In some embodiments, treated animals include domesticated animals and laboratory animals, e.g., non-mammals and mammals, for example non-human primates, rodents, pigs, dogs, and cats. In certain embodiments, the subject may be administered with additional therapeutic agents or cells.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including ASC. In another aspect, the kits and articles of manufacture may comprise a label, instructions, and packaging material, for example for treating a disorder or therapeutic indication mentioned herein.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following examples, which together with the above descriptions illustrate certain embodiments in a non-limiting fashion.

### Example 1: Culturing and Production of Adherent Placental Cells

Placenta-derived cell populations containing over 90% maternally-derived cells were prepared as described in Example 1 of International Patent Application WO 2016/098061, in the name of Esther Lukasiewicz Hagai et al, published on Jun. 23, 2016, which is incorporated herein by reference in its entirety.

Osteogenesis and adipogenesis assays were performed on placental cells prepared as described in the previous paragraph and on BM adherent cells. In osteogenesis assays, over 50% of the BM cells underwent differentiation into osteocytes, while none of the placental-derived cells exhibited signs of osteogenic differentiation. In adipogenesis assays, over 50% of the BM-derived cells underwent differentiation into adipocytes. In contrast, none of the placental-derived cells exhibited morphological changes typical of adipocytes. These experiments were performed as described in Example 2 of WO 2016/098061, which is incorporated herein by reference.

### Example 2: Culture of Placental Cells in Serum-Free Medium

Methods

Overview: The manufacturing process consisted of 3 stages, followed by downstream processing steps:

Stage 1, the intermediate cell stock (ICS) production, contains the following steps:
1. Extraction of ASCs from the placenta. Initial incubation was in serum-containing medium.
2. 2-dimensional cell growth (“2D” growth in flasks) for 3 passages in serum-free medium (typically about 4-10 population doublings after the first passage).
3. Cell concentration, formulation, filling and cryopreservation.
   Stage 2, the thawing of the ICS and initial further culture steps, contains the following step:
1. 2D cell growth of the thawed ICS in serum-free medium for 2 additional passages (3/1 and 3/2) (typically about 10-14 population doublings after thawing).
   Stage 3, the additional culture steps in the presence of serum, contains the following steps:
1. 2D cell growth of the thawed ICS in serum-containing medium for 1 additional passage. In some cases, cells were switched to serum-containing medium for the final 3 days of passage 3/2. In either case, the total number of population doublings after adding serum-containing medium was typically about 3-8.
2. 3D cell growth in a bioreactor for up to 10 additional doublings.

The downstream processing steps included harvest from flasks or bioreactor/s, cell concentration, washing, formulation, filling and cryopreservation.

The procedure included periodic testing of the growth medium for sterility and contamination.

### Production of ICS

### Step 1-1—Extraction of Adherent Stromal Cells (ASC's)

Placentas were obtained from donors up to 35 years old, who were pre-screened and determined to be negative for hepatitis B, hepatitis C, HIV-1 and HIV-2, HTLV-1 and HTLV-2, and syphilis. The donor placenta was maintained sterile and cooled until the initiation of the extraction process.

Within 36 hours of the delivery, the placenta (apart from the amnion and chorion) was placed with the maternal side facing upwards and was cut into pieces, which were washed thoroughly with isotonic buffer) containing gentamicin.
The washed pieces were incubated for 1-3 hours with collagenase and DNAse in isotonic buffer.
DMEM with 10% filtered FBS and L-Glutamine, supplemented with gentamicin, was added, and the digested tissue was coarsely filtered through a sterile stainless steel sieve and centrifuged.
The cells were suspended in culture medium, seeded in flasks, and incubated at 37° C. in a tissue culture incubator under humidified conditions supplemented with 5% Co₂.
After 2 days, cells were washed twice with Phosphate-Buffered Saline (PBS), and the culture medium was replaced with StemPro® MSC SFM XenoFree medium (serum-free and xeno-free culture medium [SFM-XF]) (ThermoFisher Scientific, catalog no. A10675-01; hereinafter “StemPro® medium”), and CellStart™ cell attachment solution was added.
Cells were incubated in StemPro® medium until the end of the first passage.

### Step 1-2—Initial 2-Dimensional Culturing

Passage 1: Cells were detached using trypsin, centrifuged, and seeded at a culture density of 3.16±0.5×10′ cells/cm² in tissue culture flasks, in gentamicin-free StemPro® medium in the presence of CellStart™.
Subsequent Passages: When the culture reached 60-90% confluence, cells were passaged as described above.

### Step 1-3—Cell Concentration, Washing, Formulation, Filling and Cryopreservation

Following the final passage, the resulting cell suspension was centrifuged and re-suspended in culture medium at a final concentration of 20-40×10⁶ cells/milliliter (mL). The cell suspension was diluted 1:1 with 2D Freezing Solution (20% DMSO, 80% FBS), and the cells were cryopreserved in 10% DMSO, 40% FBS, and 50% DMEM. The temperature was reduced in a controlled rate freezer (1° C./min down to −80° C., followed by 5° C./min down to −120° C.), and the cells were stored in a liquid nitrogen freezer to produce the ICS.

### Production of Cell Product

### Step 2-1: Additional Two-Dimensional (2D) Cell Culturing.

The ICS was thawed, diluted with and cultured in StemPro® medium until 60-90% confluence (typically 4-7 days after seeding), and cultured for 2 additional passages (referred to as passages 3/1 and 3/2 respectively; again passaging when reaching 60-90% confluence), then were harvested for seeding in the bioreactor.

### Step 2-2: Three-Dimensional (3D) Cell Growth in Bioreactor/s

Each bioreactor contained Fibra-cel® carriers (New Brunswick Scientific) made of polyester and polypropylene, and StemPro® medium.

The culture medium in the bioreactor/s was kept at the following conditions: temp: 37±1° C., Dissolved Oxygen (DO): 70±20% and pH 7.4*0.4. Filtered gases (Air, C02, N2 and 02) were supplied as determined by the control system in order to maintain the target DO and pH values.

After seeding, the medium was agitated with stepwise increases in the speed, up to 150-200 RPM by 24 hours. Perfusion was initiated several hours after seeding and was adjusted on a daily basis in order to keep the glucose concentration constant at approximately 550 mg\liter.

Cell harvest was performed at the end of the growth phase (approximately day 6). Bioreactors were washed for 1 minute with pre-warmed sterile PBS, and cells were detached.

### Step 2-3: Downstream Processing: Cell Concentration, Washing, Formulation, Filling and Cryopreservation

In some experiments, the cell suspension underwent concentration and washing, using suspension solution (5% w/v human serum albumin [HSA] in isotonic solution) as the wash buffer, and diluted 1:1 with 2×3D-Freezing solution (20% DMSO v/v and 5% HSA w/v in isotonic solution) to a final concentration of 10-20×10⁶ cells/ml, in isotonic solution containing 10% DMSO v/v and 5% HSA w/v. The temperature of the vials was gradually reduced, and the vials were stored in a gas-phase liquid nitrogen freezer.

Bone marrow migration assay. ASC were suspended in full DMEM at a concentration of 1×10⁶ cells per 4 ml. medium. An aliquot of cell suspension containing 1×10⁶ cells was added to each well of a 6-well plate and incubated for 24 hr. Cells were then washed with PBS and incubated in chemotaxis buffer (Roswell Park Memorial Institute [RPMI] with 5% albumin) for another 24 hrs., after which the CM was collected and centrifuged at 1500 rpm for 5 min, and the supernatant was retained.

Mouse BM cells were suspended at 10×10⁶ cells/ml in chemotaxis buffer, and 100 mcL (microliter) per well of the cell suspension was added to the upper compartment of 24-well Transwell® plates. 0.5 ml. CM from ASC, collected as described in the previous paragraph, was added to the lower compartment, and wells were incubated for 24 hr. at 37° C., in a 5% C02-containing incubator. The upper compartments were gently removed, medium from the lower compartments were removed, the wells were washed, and the wash buffer was combined with the removed medium. Cells were counted, and the percentage of migration was defined as the number of migrated cells divided by the total number of cells added to the well.

Results

Placental cells were extracted and expanded in serum-free (SF) medium for 3 passages. Cell characteristics of several batches were assessed (Table 1). The cells exhibited a significant ability to enhance hematopoiesis in a bone marrow migration (BMM) assay.

TABLE 1
Characteristics of placental cells expanded in SF medium.
PDL refers to population doubling level-in this case,
the number of doublings since passage 1.

| | | | Total growth | cell size | |
|---|---|---|---|---|---|
| BATCH | GROUP | Passage | (days) | (μm) | PDL |
| PD200114SFM | A | 1 | 8 | 20.3 | NA |
| | | 2 | 14 | 20.9 | 3.4 |
| | | 3 | 20 | 19.7 | 7 |
| | B | 1 | 8 | 19.5 | NA |
| | | 2 | 15 | 21.5 | 3.4 |
| | | 3 | 20 | 18.9 | 6.9 |
| PD240214SFM | A | 1 | 7 | 16.2 | NA |
| | | 2 | 14 | 20.8 | 2.7 |
| | | 3 | 20 | 19.4 | 6.4 |
| | B | 1 | 7 | 22 | NA |
| | | 2 | 14 | 18.2 | 2.1 |
| | | 3 | 20 | 19.2 | 6.1 |
| PD230414SFM | NA | 1 | 7 | NA | NA |
| | | 2 | 14 | NA | 2.3 |
| | | 3 | 19 | 16.2 | 5.7 |
| PD040514SFM | NA | 1 | 7 | NA | NA |
| | | 2 | 14 | NA | 2.7 |
| | | 3 | 18 | 15.6 | 6.5 |
| PD260514SFM | NA | 1 | 7 | NA | NA |
| | | 2 | 13 | NA | 2.9 |
| | | 3 | 17 | 15.8 | 6.6 |
| PD180814SFM | NA | 1 | 6 | NA | NA |
| | | 2 | 10 | NA | 2.1 |
| | | 3 | 16 | 16.7 | 5.3 |
| PD220914SFM | unfiltered | 1 | 8 | NA | NA |
| | | 2 | 14 | NA | 2.1 |
| | | 3 | 20 | 17 | 5.6 |
| | filtered | 1 | 8 | NA | NA |
| | | 2 | 14 | NA | 2 |
| | | 3 | 20 | 17.8 | 5.1 |
| PD271014SFM | filtered | 1 | 9 | NA | NA |
| | | 2 | 15 | NA | 2.1 |
| | | 3 | 21 | 17 | 5.1 |

| | | | |
|---|---|---|---|
| Average P 3 | 19.1 | 17.55 | 6.12 |
| % CV P 3 | 8 | 9 | 11 |

### Example 3: Two-Step Culturing Method, Utilizing Srm Followed by Serum-Containing Medium

Following extraction, placental cells are initially grown in SRM in a standard 2D tissue culture apparatus in all groups, as described in the previous Example. Three days before the end of passage 3/3, cells are transferred to DMEM+10% FBS, or DMEM+20% FBS until the end of passage 3/3, followed by bioreactor seeding and expansion in the bioreactor in the same serum-containing medium. In other experiments, cells are transferred to serum-containing medium at the time of bioreactor seeding.

### Example 4: Osteocyte and Adipose Differentiation Assays

ASC were prepared as described in Example 1. BM adherent cells were obtained as described in WO 2016/098061 to Esther Lukasiewicz Hagai and Rachel Ofir, which is incorporated herein by reference in its entirety. Osteogenesis and adipogenesis assays were performed as described in WO 2016/098061.

Osteocyte induction. Incubation of BM-derived adherent cells in osteogenic induction medium resulted in differentiation of over 50% of the BM cells, as demonstrated by positive alizarin red staining. On the contrary, none of the placental-derived cells exhibited signs of osteogenic differentiation.

Next, a modified osteogenic medium comprising Vitamin D and higher concentrations of dexamethasone was used. Over 50% of the BM cells underwent differentiation into osteocytes, while none of the placental-derived cells exhibited signs of osteogenic differentiation.

Adipocyte induction. Adipocyte differentiation of placenta- or BM-derived adherent cells in adipocyte induction medium resulted in differentiation of over 50% of the BM-derived cells, as demonstrated by positive oil red staining and by typical morphological changes (e.g. accumulation of oil droplets in the cytoplasm). In contrast, none of the placental-derived cells differentiated into adipocytes.

Next, a modified medium containing a higher indomethacin concentration was used. Over 50% of the BM-derived cells underwent differentiation into adipocytes. In contrast, none of the placental-derived cells exhibited morphological changes typical of adipocytes.

### Example 5: Further Osteocyte and Adipose Differentiation Assays

ASC were prepared as described in Examples 2-3. Adipogenesis and Osteogenesis were assessed using the STEMPRO® Adipogenesis Differentiation Kit (GIBCO, Cat# A1007001) and the STEMPRO® Osteogenesis Differentiation Kit (GIBCO, Cat# A1007201), respectively.

Results

Adipogenesis and Osteogenesis of placental cells grown in SRM or in full DMEM were tested. Groups are shown in Table 2.

TABLE 2
experimental groups

| Group | Product | Batch |
|---|---|---|
| A1 | BM derived MSC (positive control) | BM-122 |
| B1 | ASC grown in SRM | PD220914SFMS3 |
| | | R001 B1.2 |
| C1 | ASC grown in SRM | R050115 R01 |
| D1 | ASC grown in SRM | R280115 R01 |
| E1 | ASC grown in full DMEM | PT041011R36 |

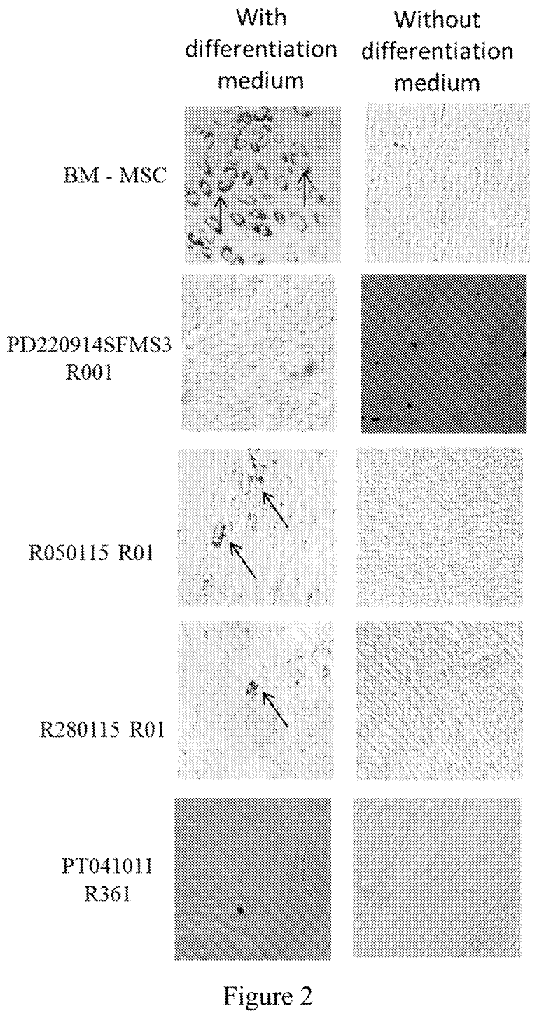

In adipogenesis assays, BM-MSCs treated with differentiation medium stained positively with Oil Red O (FIG. 2). By contrast, 2/3 of the SRM batches exhibited negligible staining, and the other SRM batch, as well as the full DMEM-grown cells, did not exhibit any staining at all, showing that they lacked significant adipogenic potential.

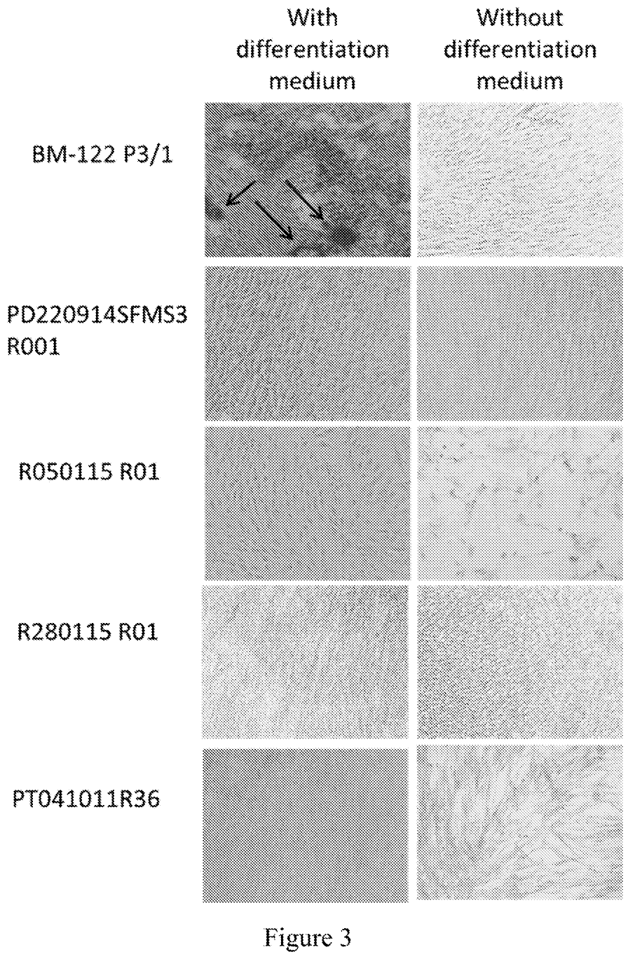

In osteogenesis assays, BM-MSCs treated with differentiation medium stained positively with Alizarin Red S (FIG. 3). By contrast, none of the placental cell batches grown in SRM or full DMEM exhibited staining, showing that they lacked significant osteogenic potential.

### Example 6: Placental ASC are Resistant to Sulfur Mustard

Methods

Placental ASC were produced as described in Examples 2-3. After cryopreservation, cells at different passages (1-4) were thawed and resuspended in DMEM+10% heat-inactivated FBS+2 mM L-glutamine. 6,735 cells per well plated with in a 96-well plate in 200 mcL volume and incubated overnight (37° C., 5% C02, humidified atmosphere), then exposed for 5 days to different concentrations of sulfur mustard (SM) diluted in ethanol, ranging from 0.2 to 1000 μM, as well as controls exposed to solvent (EtOH) alone, and blank wells with no cells. Cells were washed with PBS and XTT staining solution and incubated for about 3 h. Absorption at 450 nm with reference at 630 nm was measured. All conditions were tested in biological quadruplicates.

Results

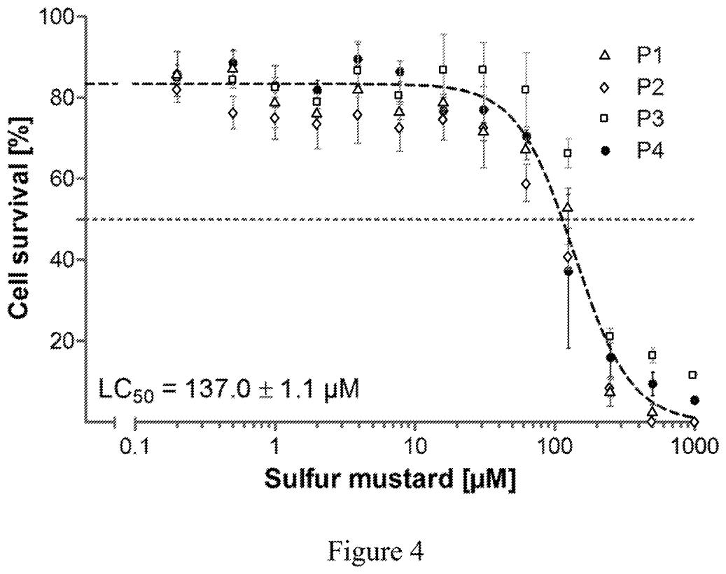

The sensitivity of placental ASC was tested by exposing ASC, produced as described in Examples 2-3, to increasing concentrations of sulfur mustard for 5 days and measuring cellular survival. Placental ASC were able to tolerate 70% more SM than BM-MSC (FIG. 4), based on the literature LC₅₀ value for BM-MSC, namely 70.7 mcM SM (Schmidt A et al.).

### Example 7: Placental ASC Improve Survival after Exposure to Sulfur Mustard

Methods

Rats were exposed to SM or EtOH using an exposure model that reliably produces pulmonary fibrosis in animals surviving 18-28 days. Fisher 344 rats were anesthetized, tracheally intubated, connected to the vapor generator and exposed to various concentrations of SM or EtOH, for 5 days. Rats were placed in a cage on a heated water blanket to recover from anesthesia, then placed in their home cage.

Results

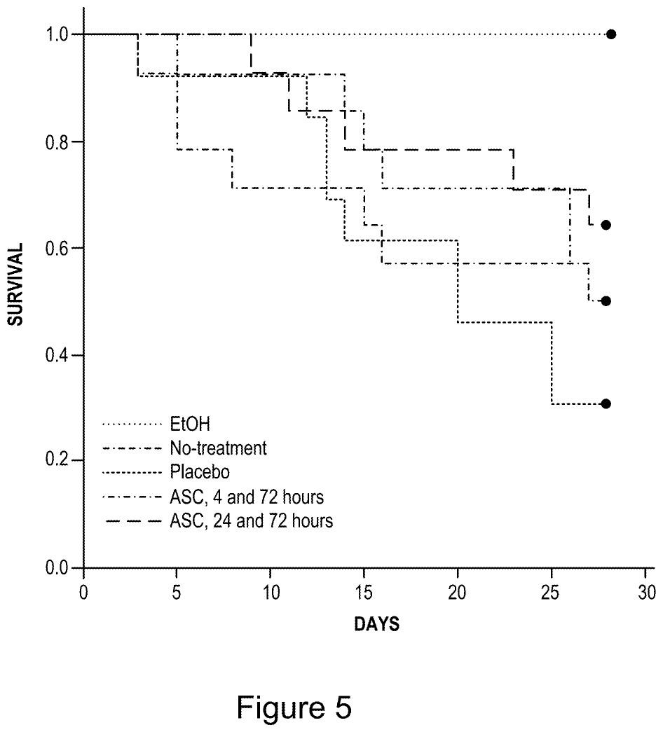

Rats were exposed to either SM or EtOH (N=6), followed by a treatment (2 dosing schedules) with placental ASC or placebo (N=15/group), at either 4 and 72 hours or 24 and 72 hours after treatment. ASC treatment imparted a significant increase in survival (FIG. 5).

### Example 8: Fetal ASC Improve Hematopoietic Parameters in Subjects with Hematopoietic Impairment

Methods

Production of CM: fetal or maternal cells were thawed and suspended in DMEM (Sigma Aldrich)+10% fetal bovine serum (FBS)+2 mM L-Glutamine, and cultured in 6 well-plate (0.5×10⁶ cells/4 ml DMEM/well) for 24 hrs in a humidified incubator (5% C02, at 37° C.). After 24 h, DMEM was removed, and cells were cultured for an additional 24 h in 1 ml RPMI 1640 medium+2 mM L-Glutamine (Biological Industries, Beit Haemek, Israel)+0.5% HSA. The CM was collected from the plate, and cell debris was discarded by 4500×g centrifugation at 4° C. for 1 min.

Cytokine level measurement: CM cytokine levels were analyzed by Bio-Plex protein assays (BIO-RAD, Hercules, Calif., USA) with BIO-RAD software data analysis, using the Luminex 100 reader (Perkin Elmer, Waltham, Mass., USA).

BM Migration assay: 10⁶ cells in 100 μl were seeded in duplicates per experimental group on the upper insert of a 5 μm 24 well-Transwell® plate with polyester permeable membrane (Corning). 0.5 ml of fetal- or maternal-derived CM or fresh RPMI medium+0.5% HSA (negative control) were added to the lower chambers of the Transwell® plate. A day later, the upper inserts were gently removed, and the migrated cells were collected from the lower chambers and quantified by CyQuant NF assay (Life Technologies Corporation, Carlsbad, Calif. USA).

Colony formation assay (CFU): 30 μl of BM cells (from a stock of 5×10⁶ cells/ml) were mixed with 1.9 ml of fetal- or maternal-derived CM or fresh RPMI medium (negative control). CM was mixed with neat methylcellulose media (R&D systems)+17% FBS. The final volume of 3.3 ml mixture was achieved with the addition of 1.4 ml methyl-cellulose to each arm. Final cell mixtures were injected with an 18-gauge needle into 6-well plate, in triplicate. Cells were incubated in 5% C02, 95% air at 37° C., for 7-12 days. Colonies which developed from single-plated HSC were inspected under a light microscope from day 5 in culture and photographed. Colonies were classified for cell type and counted and photographed.

Results

CM from fetal and maternal placental ASC were analyzed for various growth factors and chemokines. Significantly higher levels of human G-CSF, GRO, IL-6, IL-8, MCP-1, ENA-78, GM-CSF, fractalkine, MCP-3, and LIF were detected in the fetal cell CM (FIG. 6A). These results are consistent with enhanced in vivo secretion of several hematopoietic cytokines by fetal cells (as provided in PCT Publication No. WO/2016/151476, which is incorporated herein by reference in its entirety).

The fetal CM also showed a 3-fold higher induction of BM cell migration relative to maternal CM by colony formation assay (FIG. 6B). Moreover, the response was also ˜3-fold higher than the migration induced by SDF-1, an active pro-migratory chemokine used as a positive control. The assay indicated significant formation of CFU-GM, CFU-M, and BFU-E colonies.

### Example 9: ASC Reduce Pancytopenia from Mustard Exposure

To determine the ability of ASC to prevent pancytopenia following mustard exposure, sulfur mustard dissolved in DMSO is applied to the skin of mice, as described in Das L M et al, at concentrations of 10, 20 or 40 mg/kg. 12 hours later, 1×10⁶ ASC or vehicle (negative control) is administered by intramuscular or intraperitoneal injection. Development of pancytopenia is monitored by sacrificing mice prior to exposure (baseline) and 3, 7, 14, and 21 days thereafter, followed by isolation of bone marrow (BM) cells and characterization by FACS staining, BrdU cell cycle analysis, and colony-forming assay (CFA), as described in Beier F1 et al.

In other experiments, rats are anesthetized, intubated with a modified glass Pasteur pipette, and exposed to SM vapor, at 3.8 mg/kg dose (0.95 mg SM in 100 μl absolute ethanol) for 50 min, as described in Anderson D R et al. 12 hours later, surviving mice are administered 1×10⁶ ASC or vehicle by intramuscular or intraperitoneal injection. Development of pancytopenia is monitored as described hereinabove.

### Example 10: ASC Reduce Pancytopenia from Vesicant Exposure in Humans

Humans accidentally exposed to sulfur mustard are treated with supportive medical care to alleviate acute toxicity. 3-12 hours later, 150×10⁶ ASC are administered by intramuscular injection or intraosseous infusion. Occurrence and development of pancytopenia is monitored by counting neutrophils, platelets and reticulocytes each week after exposure.

### Example 11: ASC Reduce Neuroinflammation from Organophosphorus Agents

To determine the ability of ASC to modulate neuroinflammation following exposure to organophosphorus agents, rats are injected subcutaneously with 180 mcg/kg soman or intramuscularly with 0.45 milligram (mg)/kg paraoxon, followed by treatment 4, 12, or 24 hours later with 1×10⁶ ASC or vehicle (negative control) by intramuscular or intraperitoneal injection. Neural inflammation is measured prior to exposure (baseline) and 3, 7, 14, and 21 days after exposure, using the translocator protein (TSPO) tracer [¹⁸F]GE-180, as described in Sridharan S et al. Long-term effects on spatial learning and memory are quantified by the Barnes maze paradigm, also as described in Finkelstein A et al.

### Example 12: ASC Prevent OPIDP after Organophosphate Exposure

To determine the ability of ASC to modulate development of OPIDP following exposure to organophosphates, hens are administered 1.1 mg/kg, diisopropylfluorophosphate (DFP) subcutaneously, as described in Petrovid R M et al., followed by treatment 4, 12, or 24 hours later with 1×10⁶ ASC or vehicle (negative control) by intramuscular or intraperitoneal injection. OPIDP is followed as described in Petrovid R M et al.

### Example 13: ASC Prevent OPIDP after Organophosphate Exposure in Human Subjects

Humans accidentally exposed to organophosphates are treated with atropine, pralidoxime, and diazepam to alleviate acute toxicity. 3-12 hours later, 150×10⁶ ASC are administered by intramuscular injection or intraosseous infusion. OPIDP is followed by toxicological studies and electrophysiological measurements, as described in Moretto A and Lotti.

### Example 14: ASC Reduce Respiratory Damage from Toxic Chemicals

To determine the ability of ASC to prevent respiratory damage following exposure to toxic chemicals (e.g. chlorine, organophosphorus agents, or HD [undistilled mustard]), mice are exposed to chlorine gas, using an exposure chamber attached to a nebulizer (Rivkin I et al), followed by 1×10⁶ ASC or vehicle (negative control) by intramuscular or intraperitoneal injection. Respiratory damage is determined by monitoring weight gain for 2-3 weeks following exposure, as well as by monitoring blood gases, respiratory mechanics, analysis of bronchoalveolar lavage fluid, alveolar fluid clearance, and lung histology, as described in Patel et al.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### REFERENCES

### Additional References May be Cited in Text

Anderson D R, Yourick J J, Moeller R B, Petrali J P, Young G D, Byers S L, (1996). Pathologic changes in rat lungs following acute sulfur mustard inhalation. Inhal. Toxicol. 8:285-297.
Bader A M, Klose K, Bieback K, et al. Hypoxic preconditioning increases survival and pro-angiogenic capacity of human cord blood mesenchymal stromal cells in vitro. PLoS One. 2015; 10: e0138477.
Barros S, Dehez S, Arnaud E, et al. Aging-related decrease of human ASC angiogenic potential is reversed by hypoxia preconditioning through ROS production. Mol Ther. 2013; 21: 399-408.
Beier F1 et al, Conditional TRF1 knockout in the hematopoietic compartment leads to bone marrow failure and recapitulates clinical features of dyskeratosis congenita. Blood. 2012 Oct. 11; 120(15):2990-3000.
Boyette L B, Creasey O A, Guzik L, et al. Human bone marrow-derived mesenchymal stem cells display enhanced clonogenicity but impaired differentiation with hypoxic preconditioning. Stem Cells Transl Med. 2014; 3: 241-54.
Chapman S et al, Sarin-induced brain damage in rats is attenuated by delayed administration of midazolam. Neurotoxicology. 2015 July; 49:132-8.
Chase L G et al, Development and Characterization of a Clinically Compliant Xeno-Free Culture Medium in Good Manufacturing Practice for Human Multipotent Mesenchymal Stem Cells. Stem Cells Transl Med. 2012 October; 1(10): 750-758.
Clayton A et al, Analysis of antigen presenting cell derived exosomes, based on immuno-magnetic isolation and flow cytometry. J Immunol Methods. 2001; 247(1-2):163-74.
Conde-Vancells et al. (2010) Candidate biomarkers in exosome-like vesicles purified from rat and mouse urine samples. Proteomics Clin. Appl. 4, 416-425.
Crescitelli R et al, Distinct RNA profiles in subpopulations of extracellular vesicles: apoptotic bodies, microvesicles and exosomes. J Extracell Vesicles. 2013 Sep. 12; 2.
Das L M et al, Early indicators of survival following exposure to mustard gas: Protective role of 25(OH)D. Toxicol Lett. 2016 Apr. 25; 248:9-15.
Dominici et al. Minimal criteria for defining multipotent mesenchymal stromal cells. The International Society for Cellular Therapy position statement. Cytotherapy. 2006; 8(4):315-7.
Duijvestein M, Wildenberg M E, Welling M M, et al. Pretreatment with interferon-gamma enhances the therapeutic activity of mesenchymal stromal cells in animal models of colitis. Stem Cells. 2011; 29: 1549-58.
Finkelstein A et al, Immunomodulation by poly-Y E reduces organophosphate-induced brain damage. Brain Behav Immun. 2012 January; 26(1): 159-69.
Golderman V et al, The Organophosphate Paraoxon and Its Antidote Obidoxime Inhibit Thrombin Activity and Affect Coagulation In Vitro. PLoS One. 2016 Sep. 30; 11(9):e0163787.
Grauer E et al, Single whole-body exposure to sarin vapor in rats: long-term neuronal and behavioral deficits. Toxicol Appl Pharmacol. 2008 Mar. 1; 227(2):265-74.
Grønningswter I S et al, Bronchiolitis obliterans syndrome in adults after allogeneic stem cell transplantation-pathophysiology, diagnostics and treatment. Expert Rev Clin Immunol. 2017 Jan. 17:1-17. doi: 10.1080/1744666X.2017.1279053. [Epub ahead of print]
Hu C and Li L. Preconditioning influences mesenchymal stem cell properties in vitro and in vivo. J Cell Mol Med. 2018 Feb. 1. doi: 10.1111/jcmm.13492. [Epub ahead of print]
Imamura. Physiological Functions and Underlying Mechanisms of Fibroblast Growth Factor (FGF) Family Members: Recent Findings and Implications for Their Pharmacological Application. Biological and Pharmaceutical Bulletin, Vol. 37(7):1081-1089 (2014).
Kannagi R et al, Stage-specific embryonic antigens (SSEA-3 and -4) are epitopes of a unique globo-series ganglioside isolated from human teratocarcinoma cells. EMBO J. 1983; 2(12):2355-61.
Katalan S et al, Magnesium sulfate treatment against sarin poisoning: dissociation between overt convulsions and recorded cortical seizure activity. Arch Toxicol. 2013 February; 87(2):347-60.
Kheirandish M, Gavgani S P, Samiee S. The effect of hypoxia preconditioning on the neural and sternness genes expression profiling in human umbilical cord blood mesenchymal stem cells. Transfus Apher Sci. 2017; 56: 392-9.
Kim K J, Joe Y A, Kim M K, et al. Silica nanoparticles increase human adipose tissue-derived stem cell proliferation through ERK1/2 activation. Int J Nanomedicine. 2015; 10: 2261-72
Kim Y S, Noh M Y, Cho K A, et al. Hypoxia/reoxygenation-preconditioned human bone marrow-derived mesenchymal stromal cells rescue ischemic rat cortical neurons by enhancing trophic factor release. Mol Neurobiol. 2015; 52: 792-803.
Kinzebach S and Bieback K. Expansion of Mesenchymal Stem/Stromal cells under xenogenic-free culture conditions. Adv Biochem Eng Biotechnol. 2013; 129:33-57.
Koga K., Matsumoto K., Akiyoshi T., Kubo M., Yamanaka N., Tasaki A., Nakashima H., Nakamura M., Kuroki S., Tanaka M., Katano M. (2005) Purification, characterization and biological significance of tumor-derived exosomes. Anticancer Res. 25, 3703-3707.
Lan Y W, Theng S M, Huang T T, et al. Oncostatin M-preconditioned mesenchymal stem cells alleviate bleomycin-induced pulmonary fibrosis through paracrine effects of the hepatocyte growth factor. Stem Cells Transl Med. 2017; 6: 1006-17.
Li D, Liu Q, Qi L, et al. Low levels of TGF-beta1 enhance human umbilical cord-derived mesenchymal stem cell fibronectin production and extend survival time in a rat model of lipopolysaccharide-induced acute lung injury. Mol Med Rep. 2016; 14: 1681-92.
Liu J, Hao H, Xia L, et al. Hypoxia pretreatment of bone marrow mesenchymal stem cells facilitates angiogenesis by improving the function of endothelial cells in diabetic rats with lower ischemia. PLoS One. 2015; 10: e0126715.
Liu X, Duan B, Cheng Z, et al. SDF-1/CXCR4 axis modulates bone marrow mesenchymal stem cell apoptosis, migration and cytokine secretion. Protein Cell. 2011; 2: 845-54.
Massa C B et al, Acute chlorine gas exposure produces transient inflammation and a progressive alteration in surfactant composition with accompanying mechanical dysfunction. Toxicol Appl Pharmacol. 2014 Jul. 1; 278(1):53-64.
Mathias R A et al, Isolation of extracellular membranous vesicles for proteomic analysis. Methods Mol Biol. 2009; 528:227-42.
McElroy C S and Day B J. Antioxidants as potential medical countermeasures for chemical warfare agents and toxic industrial chemicals. Biochem Pharmacol. 2016 Jan. 15; 100:1-11.
Mimura et al, Growth factor-defined culture medium for human mesenchymal stem cells. Int. J. Dev. Biol. 55: 181-187 (2011).
Moretto A and Lotti M, Poisoning by organophosphorus insecticides and sensory neuropathy. J Neurol Neurosurg Psychiatry. 1998 April; 64(4):463-8.
Najafi R, Sharifi A M. Deferoxamine preconditioning potentiates mesenchymal stem cell homing in vitro and in streptozotocin-diabetic rats. Expert Opin Biol Ther. 2013; 13: 959-72.
Noiseux N, Borie M, Desnoyers A, et al. Preconditioning of stem cells by oxytocin to improve their therapeutic potential. Endocrinology. 2012; 153: 5361-72.
Patel B V et al, Resolution of acute lung injury and inflammation: a translational mouse model. Eur Respir J. 2012 May; 39(5):1162-70.
Petrović R M et al, The treatment of delayed polyneuropathy induced by diisopropylfluorophosphate in hens. Pharmazie. 2000 June; 55(6):454-5.
Pourjafar M, Saidijam M, Mansouri K, et al. All-trans retinoic acid preconditioning enhances proliferation, angiogenesis and migration of mesenchymal stem cell in vitro and enhances wound repair in vivo. Cell Prolif. 2017; 50: e12315
Qiu Y, Guo J, Mao R, et al. TLR3 preconditioning enhances the therapeutic efficacy of umbilical cord mesenchymal stem cells in TNBS-induced colitis via the TLR3-Jagged-1-Notch-1 pathway. Mucosal Immunol. 2017; 10: 727-42.
Rajaraman G et al, Optimization and scale-up culture of human endometrial multipotent mesenchymal stromal cells: potential for clinical application. Tissue Eng Part C Methods. 2013 Jan.; 19(1):80-92.
Rivkin I et al, Treatment of respiratory damage in mice by aerosols of drug-encapsulating targeted lipid-based particles. J Control Release. 2016 Apr. 5. pii: SO168-3659(16)30185-7.
Rosman Y et al, Using MRI for the assessment of paraoxon-induced brain damage and efficacy of antidotal treatment. J Appl Toxicol. 2012 June; 32(6):409-16.
Schmidt A et al., Mesenchymal stem cells are highly resistant to sulfur mustard. Chem Biol Interact. 2013 Dec. 5; 206(3):505-11
Shrot S et al, Early brain magnetic resonance imaging can predict short and long-term outcomes after organophosphate poisoning in a rat model. Neurotoxicology. 2015 May; 48:206-16.
Sridharan S et al, Comparative Evaluation of Three TSPO PET Radiotracers in a LPS-Induced Model of Mild Neuroinflammation in Rats. Mol Imaging Biol. 2017 February; 19(1):77-89.
Sun J, Wei Z Z, Gu X, et al. Intranasal delivery of hypoxia-preconditioned bone marrow-derived mesenchymal stem cells enhanced regenerative effects after intracerebral hemorrhagic stroke in mice. Exp Neurol. 2015; 272: 78-87.
Tang Y, Cai B, Yuan F, et al. Melatonin pretreatment improves the survival and function of transplanted mesenchymal stem cells after focal cerebral ischemia. Cell Transplant. 2014; 23: 1279-91.
Th\tilde{e}ry C et al, Isolation and characterization of exosomes from cell culture supernatants and biological fluids. Curr Protoc Cell Biol. 2006 April; Chapter 3:Unit 3.22. doi: 10.1002/0471143030.cb0322s30.
Urnukhsaikhan E, Cho H, Mishig-Ochir T, et al. Pulsed electromagnetic fields promote survival and neuronal differentiation of human B M-MSCs. Life Sci. 2016; 151: 130-8.
Waszak P, Alphonse R, Vadivel A, Ionescu L, Eaton F, Thebaud B. Preconditioning enhances the paracrine effect of mesenchymal stem cells in preventing oxygen-induced neonatal lung injury in rats. Stem Cells Dev. 2012; 21(15):2789-2797.
Winkler T et al., Immunomodulatory placental-expanded, mesenchymal stromal cells improve muscle function following hip arthroplasty. J Cachexia Sarcopenia Muscle. 2018 October; 9(5):880-897. doi: 10.1002/jcsm.12316.
Xia W, Zhang F, Xie C, et al. Macrophage migration inhibitory factor confers resistance to senescence through CD74-dependent AMPK-FOXO3a signaling in mesenchymal stem cells. Stem Cell Res Ther. 2015; 6: 82.
Yin K, Zhu R, Wang S, et al. Low-level laser effect on proliferation, migration, and anti-apoptosis of mesenchymal stem cells. Stem Cells Dev. 2017; 26: 762-75.

1. A method of reducing a morbidity in a subject exposed to a vesicant, comprising administering to said subject a pharmaceutical composition comprising adherent stromal cells (ASC), thereby reducing a morbidity in a subject exposed to a vesicant.

2. The method of claim 1, wherein said morbidity comprises chronic inflammation, pulmonary fibrosis, pulmonary hypertension, or pancytopenia.

3. A method of reducing a mortality in a subject exposed to a vesicant, comprising administering to said subject a pharmaceutical composition comprising adherent stromal cells (ASC), thereby reducing a mortality in a subject exposed to a vesicant.

4. The method of claim 1, wherein said vesicant is selected from Lewisite, a mustard compound, and an organic arsenic compound.

5. A method of reducing central nervous system (CNS) damage in a subject exposed to an organophosphate agent, comprising administering to said subject a pharmaceutical composition comprising adherent stromal cells (ASC), thereby reducing CNS damage in a subject exposed to an organophosphate agent.

6. The method of claim 5, wherein said CNS damage results from CNS inflammation.

7. The method of claim 5, wherein said CNS damage comprises learning deficits, memory impairment, insomnia, or a personality alteration.

8. (canceled)

9. (canceled)

10. The method of claim 1, wherein said ASC have been incubated in a 3D culture apparatus.

11. The method of claim 10, further comprising harvesting said ASC by removing said ASC from said 3D culture apparatus.

12. The method of claim 10, wherein said ASC have been incubated in a 2D adherent-cell culture apparatus, prior to said incubation in a 3D culture apparatus.

13. The method of claim 10, wherein said 3D culture apparatus comprises a microcarriers disposed within a bioreactor.

14. The method of claim 10, wherein said 3D culture apparatus comprises a synthetic adherent material, wherein said synthetic adherent material is a fibrous matrix.

15. (canceled)

16. The method of claim 14, wherein said synthetic adherent material is selected from the group consisting of a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber.

17. (canceled)

18. The method of claim 1, wherein said administering comprises:
a. administering to the subject a first pharmaceutical composition, comprising ASC from a first donor; and
b. administering to said subject, at least 7 days after step a), a second pharmaceutical composition comprising allogeneic ASC from a second donor, wherein said second donor differs from said first donor in at least one allele group of human leukocyte antigen (HLA)-A or human leukocyte antigen (HLA)-B,

19. (canceled)

20. The method of claim 1, wherein said ASC originate from placenta tissue.

21. The method of claim 20, wherein said ASC express a marker selected from the group consisting of CD73, CD90, CD29 and CD105.

22. The method of claim 20, wherein said ASC do not express a marker selected from the group consisting of CD3, CD4, CD11b, CD14, CD19, and CD34.

23. The method of claim 20, wherein said ASC do not express a marker selected from the group consisting of CD3, CD4, CD34, CD39, and CD106.

24-31. (canceled)

32. The method of claim 1, wherein the cells are administered intramuscularly, intravenously, subcutaneously, or intraperitoneally.

33. (canceled)

34. The method of claim 1, wherein the cells are administered intratracheally, intrathecally, by inhalation, or intranasally.
